(19) United States
(12) Patent Application Publication
(10) Pub. No.: US 2025/0241672 A1
(43) Pub. Date: Jul. 31, 2025

(54) PIVOT JOINTS FOR SURGICAL CUTTING DEVICES, AND SYSTEMS AND METHODS THEREOF

(71) Applicant: Distalmotion SA, Epalinges (CH)

(72) Inventors: Romain NICOLET, Lausanne (CH); James CROZIER, Cully (CH)

(21) Appl. No.: 19/183,500

(22) Filed: Apr. 18, 2025

Related U.S. Application Data
(63) Continuation of application No. PCT/IB2023/060543, filed on Oct. 19, 2023.
(60) Provisional application No. 63/380,201, filed on Oct. 19, 2022.

(51) Int. Cl. A61B 17/3201

(52) U.S. Cl. CPC A61B 17/3201

(57) ABSTRACT

The present disclosure relates to surgical robotic systems having surgical scissors. In some embodiments, the surgical scissors include independently actuatable cutting members, where each cutting member is configured to rotate about an axis of a pin to perform a cutting operation. In some embodiments, the surgical scissors include an integrated spring. In some embodiments, the surgical scissors include cutting members that are configured to rotate about one or more axes while being constrained in rotation about another axis. In some embodiments, the surgical scissors include multiple curvatures, in which one or more curvatures can maintain an opening angle of the scissors.

### CROSS-REFERENCE TO RELATED APPLICATIONS

This application claims the benefit of U.S. Provisional Application No. 63/380,201, filed Oct. 19, 2022, the content of which is hereby incorporated by reference in its entirety.

### TECHNICAL FIELD

This application generally relates to surgical robotic systems, for example, an end effector with spring-based scissor blades for a surgical instrument.

### BACKGROUND

Surgical scissors are generally used to cut human tissue during surgical procedures by bringing together the cutting edges of the blades of the surgical scissors. The cutting edges may be brought together by rotating the blades around an axis of rotation. However, typical surgical scissors often experience changes in shear angle, which results in uneven or ineffective cuts.

Moreover, surgical scissors that are used in traditional laparoscopy often have tabs that extend proximally away from the pivot point of the surgical scissor, proximal to the cutting blades. These tabs are curved toward each other, such that when held together, the tabs act as springs to thereby provide the reaction force of the blades. While manual full-sized scissors often have a gentle curvature of the blades which can be used to provide the reaction force, this is not possible in surgical scissors because surgical scissors do not have enough length for the gentle curve used by manual scissors. Moreover, it may not be desirable to design robotic surgical scissors in the same way as laparoscopic scissors as such tabs would elongate the instrument tip and reduce dexterity.

Therefore, the blades of some surgical scissor assemblies cannot cut effectively, and for some other surgical scissor assemblies, the blades may cut unevenly due to varying shear angles. Accordingly, improved surgical scissors that can be used in robotic surgery are desirable.

### SUMMARY

The present disclosure relates generally to systems, devices, and methods for providing an apparatus for use with a surgical instrument, such as a cutting end effector for use with a surgical instrument.

In some embodiments, an apparatus may include a first cutting member and a second cutting member, where each of the first and second cutting members have a cutting edge and a mounting end that defines an oblong opening. A pin may extend through the oblong openings of each of the mounting ends of the first and second cutting members. Each of the first and second cutting members may be configured to independently rotate about a first axis defined by the pin and a second axis perpendicular to the first axis, while being constrained in rotation about a third axis perpendicular to the first and second axes, such that a shear angle between the first and second cutting edges of the first and second cutting members may remain constant. In some embodiments, the shear angle may be between about 160 degrees to about 180 degrees. Each of the first and second cutting members may have a curved section. The cutting edge of the first cutting member may contact the cutting edge of the second cutting member at a contact point that translates along the respective cutting edges when one or more of the first and second cutting members rotate about the first axis. In some embodiments, the first and second cutting members may be configured to translate along the first axis.

Each oblong opening may have a first lateral dimension that is smaller than a second later dimension. The second axis may be parallel to the first lateral dimension. The first lateral dimension may be substantially equal to an outer diameter of the pin. Each of the mounting ends of the first and second cutting members may further define a circular opening that is connected to the oblong opening. The pin may be configured to extend through the circular opening and the oblong opening of each of the mounting ends of the first and second cutting members. In some embodiments, the first lateral dimension of the oblong opening may be substantially equal to a diameter of the circular opening. The first lateral dimension of the oblong opening and the diameter of the circular opening may be substantially equal to an outer diameter of the pin. The oblong openings may be disposed on inner sides of the first and second cutting members that face one another, and the circular openings may be disposed on outer sides of the first and second cutting members.

The apparatus may further include a first spring coupled to the first cutting member and a second spring coupled to the second cutting member. The first and second springs may be configured to press the first and second cutting members against one another. Each of the first and second springs may comprise a Belleville spring. Each of the first and second springs may be configured to allow the respective one of the first and second cutting members to translate along the first axis.

In some embodiments, an apparatus may include a first cutting member including a first cutting edge and a first mounting end defining a first oblong opening, a second cutting member including a second cutting edge and a second mounting end defining a second oblong opening aligned with the first oblong opening, and a cylindrical pin defining an x-axis. The cylindrical pin may extend through the first and second oblong openings. Each of the first and second oblong openings may include a first lateral dimension substantially equal to a diameter of the cylindrical pin and a second lateral dimension greater than the diameter of the cylindrical pin. Each of the first and second cutting members may be configured to (1) independently rotate about the x-axis and about a z-axis that may be parallel to the first lateral dimension of the first and second oblong openings and (2) independently translate along the x-axis, while being constrained in rotation about a y-axis that may be perpendicular to the x-axis and the z-axis. A ratio of the first lateral dimension to the second lateral dimension of the first and second oblong openings may be configured to allow a rotation about the z-axis to accommodate translation of a contact point between the first and second cutting members.

Each of the first and second cutting members may have a curved section. Each of the first and second cutting members may be configured to rotate about one or more of the x-axis and the z-axis and translate along the x-axis simultaneously. The first cutting edge may contact the second cutting edge at a contact point that translates along the first and second cutting edges when one or both of the first and second cutting members rotate about the x-axis. In some embodiments, this may be facilitated by a rotation of one or both of the first and second cutting members about the z-axis.

The apparatus may further include a first spring coupled to the first cutting member and a second spring coupled to the second cutting member. The first and second springs may be configured to press the first and second cutting members against one another. Each of the first and second springs may comprise a Belleville spring.

In some embodiments, an apparatus may include a first cutting member and a second cutting member, where each of the first and second cutting members may include a cutting edge, a coupled proximal end, and a free distal end, and a pin extending through the coupled proximal end of each of the first and second cutting members such that each of the first and second cutting members may be configured to independently rotate about an axis of the pin. The cutting edge of each of the first and second cutting members may have a proximal portion including a curvature having a first radius of curvature and a distal portion including a curvature having a second radius of curvature smaller than the first radius of curvature. Thus, an opening angle

between the cutting edges of each of the first and second cutting members may remain constant as the free distal ends of the first and second cutting members may be rotated toward one another to form an incision in a cutting plane. The opening angle may be between about 30 degrees to about 40 degrees, e.g., throughout the whole cutting range.

The distal portion of each of the first and second cutting members may include between about 25% to about 40% of a length of the respective first or second cutting member. The distal portion of each of the first and second cutting members may include the free distal end of the respective first or second cutting member. The curvature of each of the proximal and distal portions may curve in a direction away from the cutting plane. The curvature of each of the proximal and distal portions may be a first curvature, and each of the proximal and distal portions may include a second curvature in a direction parallel to the cutting plane.

In some embodiments, the apparatus may include a first spring coupled to the first cutting member and a second spring coupled to the second cutting member. The first and second springs may be configured to press the first and second cutting members against one another. Each of the first and second springs may comprise a Belleville spring.

The coupled proximal end of each of the first and second cutting members may define an oblong opening. The pin may extend through the oblong opening. The oblong opening may have a first lateral dimension that may be smaller than a second lateral dimension. The first lateral dimension may be substantially equal to an outer diameter of the pin. The axis of the pin may be a first axis, and each of the first and second cutting members may be configured to independently rotate about the first axis and a second axis that is perpendicular to the first axis while being constrained in rotation about a third axis that is perpendicular to the first and second axes, such that a shear angle between the cutting edges of each of the first and second cutting members may remain constant. The shear angle may be between about 160 degrees to about 180 degrees.

According to embodiments, apparatuses described herein can provide a constant cutting angle. The apparatus can include first and second cutting members that form a cutting angle while performing a cut. The cutting angle may be defined at the point of contact of the cutting edges of the cutting members. The curvature of one of the cutting members, e.g., a first cutting member, may be a predetermined curvature, whereas the curvature of the other cutting member, e.g., second cutting member, may be defined depending on the predetermined curvature of the first cutting member so as to maintain a constant cutting angle between the respective cutting edges of the first and second cutting members. In embodiments, the cutting edge of the first cutting member has a constant curvature radius in a xy-plane (e.g., cutting plane), whereas cutting edge of the second cutting member has portions each with a curvature radius that is chosen so as to maintain a constant cutting angle between the respective cutting edges of the first and second cutting members. The cutting edge of the second cutting member comprises at least two portions named proximal and distal portions, each having a determined radius of curvature. The number of portions may be chosen so as to maintain the cutting angle as constant (or substantially constant) when the first and second cutting members are rotated toward one another. In some embodiments, the number of portions can be between 1 and 10 portions, including all values and sub-ranges therebetween. Thus, a cutting angle between the cutting edges of each of the first and second cutting members may remain constant as the free distal ends of the first and second cutting members may be rotated toward one another to form an incision in a cutting plane. The cutting angle may be between about 1 degree to about 5 degrees, including all values and sub-ranges therebetween.

In some embodiments, the end effector may include a first scissor blade having a first mounting body configured to be actuated to rotate the first scissor blade about an axis of the end effector, and a first blade having a first root portion coupled to the first mounting body and a first cutting portion extending distally from the first root portion. The first root portion may include a first spring integrally formed with the first blade. In addition, the end effector may include a second scissor blade having a second mounting body configured to be actuated to rotate the second scissor blade about the axis of the end effector, and a second blade having a second root portion coupled to the second mounting body and a second cutting portion extending distally from the second root portion. The second root portion also may include a second spring integrally formed with the second blade. Moreover, the first and second mounting bodies are configured to be independently actuatable such that actuation of the first and second mounting bodies in opposition directions causes actuation of the first and second scissor blades in an open and close degree of freedom.

At least one of the first and second springs may include a U-shaped spring, and the first and second blades may have a predetermined curvature. The first and second springs may not extend proximally beyond the axis of the end effector. In addition, the first and second springs are configured to provide a relatively consistent reaction force between the first and second blades in the open and close degree of freedom. The first mounting body may be integrally formed with the first blade, and the second mounting body may be integrally formed with the second blade. Actuation of the first and second mounting bodies in the same direction may cause actuation of the first and second scissor blades in a pitch degree of freedom.

The second mounting body may be concentrically aligned with the first mounting body. Additionally, the first and second mounting bodies may be configured to be independently actuatable via first and second force transmitting elements, respectively. For example, the first and second force transmitting elements may be cables. The first mounting body may have a first groove sized and shaped to receive the first force transmitting element, and the second mounting body may have a second groove sized and shaped to receive the second force transmitting element. Moreover, the first mounting body may include a first crimp configured to secure the first force transmitting element to the first groove, and the second mounting body may include a second crimp configured to secure the second force transmitting element to the second groove. The end effector further may include a frame having a proximal region and a distal region configured to rotatably receive the first and second mounting bodies. The frame may include a pin configured to permit rotation of the first and second mounting bodies about the axis of the end effector. In addition, the proximal region of the frame may be configured to be actuated to cause actuation of the first and second scissor blades in a yaw degree of freedom.

In some embodiments, a proximal portion of the end effector may include one or more knobs sized and shaped to be received by one or more corresponding openings disposed on a distal region of an instrument shaft of the surgical instrument, the distal region of the instrument shaft comprising a flexible flap configured to secure the one or more knobs within the one or more corresponding openings to thereby secure the end effector to the instrument shaft. The one or more knobs may have a geometry such that movement of the one or more knobs proximally relative to the flexible flap causes the flexible flap to transition between a radially expanded state, where the one or more knobs are permitted to move toward the one or more corresponding grooves, and a collapsed state, where the flexible flap secures the one or more knobs within the one or more corresponding grooves.

Accordingly, the end effector for use with the surgical instrument may include a pair of independently actuatable scissor blades, each scissor blade comprising a mounting body configured to be actuated to rotate the respective scissor blade of the pair of independently actuatable scissor blades about an axis of the mounting body, and a blade comprising a root portion coupled to the mounting body and a cutting portion extending distally from the root portion. The root portion may include a spring integrally formed with the blade, which is configured to provide a relatively consistent reaction force between the pair of independently actuatable scissor blades upon actuation of the pair of independently actuatable scissor blades. Accordingly, actuation of the pair of independently actuatable scissor blades in opposition directions causes actuation of the blades of the pair of independently actuatable scissor blades in an open and close degree of freedom.

In accordance with another aspect of the present disclosure, a method for actuating an end effector of a surgical instrument is provided. The method may include: rotating a first mounting body of a first scissor blade of the end effector to actuate a first blade extending from the first mounting body via a first root portion, the first root portion comprising a first U-shaped spring integrally formed with the first blade; and rotating a second mounting body of a second scissor blade of the end effector to actuate a second blade extending from the second mounting body via a second root portion, the second root portion comprising a second U-shaped spring integrally formed with the second blade, wherein rotation of the first and second mounting bodies in opposite directions causes actuation of the first and second scissor blades in an open and close degree of freedom, such that the first and second U-shaped springs provide a relatively consistent reaction force between the first and second blades.

In accordance with another aspect of the present disclosure, a surgical instrument having the end effector, and a surgical robot system having the surgical instrument are provided. For example, an instrument shaft of the surgical instrument may include one or more openings disposed on a distal region of the instrument shaft, and one or more flexible flaps distal to the one or more openings, such that the one or more flexible flaps extend from a distal end of the instrument shaft toward the one or more openings. The surgical instrument further may include an end effector configured to be removably coupled to the distal region of the instrument shaft, such that a proximal portion of the end effector may include one or more knobs sized and shaped to be received by the one or more openings of the instrument shaft. In addition, the one or more knobs may have a geometry such that movement of the one or more knobs proximally relative to the one or more flexible flaps causes the one or more flexible flaps to transition between a radially expanded state, where the one or more knobs are permitted to move toward the one or more openings, and a collapsed state, where the one or more flexible flaps secure the one or more knobs within the one or more openings to thereby secure the end effector to the instrument shaft.

In addition, the instrument shaft may include one or more openings disposed on a proximal region of the instrument shaft, and one or more flexible flaps proximal to the one or more openings. The one or more flexible flaps may extend from a proximal end of the instrument shaft toward the one or more openings at the proximal region of the instrument shaft. Accordingly, the system further may include an instrument hub configured to be removably coupled to a proximal region of the instrument shaft, such that the instrument hub may include one or more hub knobs sized and shaped to be received by the one or more openings at the proximal region of the instrument shaft. Moreover, the one or more hub knobs of the instrument hub may have a geometry such that movement of the one or more hub knobs distally relative to the one or more flexible flaps causes the one or more flexible flaps to transition between a radially expanded state, where the one or more hub knobs are permitted to move toward the one or more openings, and a collapsed state, where the one or more flexible flaps secure the one or more hub knobs within the one or more openings to thereby secure the instrument hub to the instrument shaft.

### BRIEF DESCRIPTION OF THE DRAWINGS

FIG. 1 schematically depicts a surgical robotic system, according to embodiments.

FIG. 2 schematically depicts a manipulator of a surgical robotic system, according to embodiments.

FIG. 3 schematically depicts the instrument of the surgical robotic system of FIG. 2, according to embodiments.

FIG. 4 schematically depicts the end effector of the instrument of FIG. 3, according to embodiments.

FIG. 5 depicts a surgical robotic system, according to embodiments.

FIG. 6 depicts a detailed view of an instrument coupling of a surgical robotic system, according to embodiments.

FIG. 7 depicts a surgical instrument of a surgical robotic system, according to embodiments.

FIG. 8A depicts a perspective view of the end effector of the surgical instrument of FIG. 7, according to embodiments. FIG. 8B depicts a top view of the end effector of the surgical instrument of FIG. 7, according to embodiments. FIG. 8C depicts a first cutting portion of the end effector of the surgical instrument of FIG. 7, according to embodiments.

FIGS. 9A and 9B depict cross-sectional views of the end effector of FIGS. 8A and 8B, according to embodiments.

FIGS. 10A-10C depict actuation of an end effector around an axis of rotation, according to embodiments.

FIG. 11A depicts a coupling mechanism for coupling an end effector to a surgical instrument shaft, according to embodiments. FIG. 11B depicts the coupling mechanism of FIG. 11A, according to embodiments. FIG. 11C depicts a proximal end of the surgical instrument shaft of FIGS. 11A and 11B.

FIG. 12 depicts a variation of a surgical instrument of a surgical robotic system, according to embodiments.

FIG. 13 depicts the surgical instrument of FIG. 12 without the shaft, according to embodiments.

FIG. 14A depicts axes of motion of an end effector, according to embodiments. FIG. 14B depicts an axis of rotation of an end effector, according to embodiments. FIG. 14C depicts another axis of rotation of an end effector, according to embodiments.

FIG. 15A depicts an end effector in a closed configuration, according to embodiments.

FIG. 15B depicts an end effector in an open configuration, according to embodiments.

FIG. 16 depicts axes of an end effector, according to embodiments.

FIG. 17A depicts an opening of an end effector, according to embodiments. FIGS. 17B and 17C depict dimensions of the opening of the end effector of FIG. 17A, according to embodiments.

FIG. 18 depicts a pin within an opening of an end effector, according to embodiments.

FIGS. 19A and 19B depict an opening angle of an end effector, according to embodiments.

FIGS. 20A and 20B depict an alternative opening angle of an end effector, according to embodiments.

### DETAILED DESCRIPTION

The present disclosure relates to cutting devices for use with robotic surgical systems. Systems, devices, and methods described herein allow a surgical robotic system to perform effective and substantially even cuts during a surgical procedure. For example, the cutting devices can be surgical scissors having scissor blades configured to cut tissue. The surgical scissors can include one or more springs configured to press the scissor blades together. The one or more springs can provide freedom to the scissor blades to translate along and/or rotate about one or more axes. The scissor blades can define one or more openings configured to receive a pin. The one or more openings of the scissor blades can have a shape that allows the scissor blades to independently rotate about one or more axes defined by the pin while being constrained in rotation about another axis defined by the pin. Combined with the effects of the one or more springs, the constrained rotation facilitated by the one or more openings can advantageously provide a constant or substantially constant shear angle as the scissor blades rotate about one or more axes. The constant shear angle can provide effective and substantially even cuts during use of the surgical scissors.

According to embodiments, the surgical scissors described herein can also provide a constant or substantially constant opening angle. The opening angle can be defined at the point of contact between the cutting edges of the scissor blades. The point of contact can traverse the length of the scissor blades as the scissor blades transition from an open configuration to a closed configuration (e.g., during a cutting process). Associated with the opening angle can be a slice-push ratio. The slice-push ratio can refer to the magnitude of cutting the material between the scissor blades to the magnitude of pushing the material between the scissor blades. The slice-push ratio can increase as the opening angle decreases, which can result in less even and/or effective cuts. In contrast, the constant opening angle maintained by the surgical scissors described herein can facilitate a constant slice-push ratio. The constant slice-push ratio can be optimized to facilitate the desired magnitude of cutting material to pushing material, which can result in optimal cutting performance (e.g., effective and substantially even cuts). The constant slice-push ratio can be pre-determined by the design of the scissor blades. According to embodiments, the scissor blades can include one or more curved portions, which may facilitate a constant or substantially constant opening angle and a corresponding constant or substantially constant slice-push ratio.

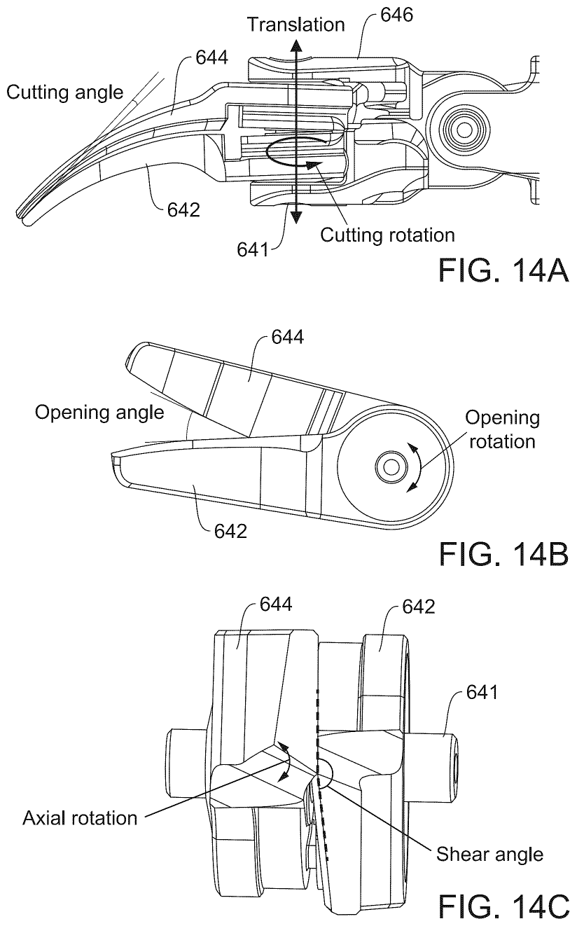

According to embodiments, the surgical scissors described herein can also provide a constant or substantially constant cutting angle. The angle formed between the first and second cutting members while performing the cut can be referred to as a cutting angle, as further described below with reference to FIG. 14A. The cutting angle may be defined at the point of contact of the cutting edges of the cutting members. The curvature of one of the cutting members, e.g., first cutting member, may be predetermined, whereas the curvature of the other cutting member, e.g., second cutting member, may be defined depending on the predetermined curvature of the first cutting member so as to maintain a constant or substantially constant cutting angle between the respective cutting edges of the first and second cutting members. In embodiments, the cutting edge of the first cutting member has a constant curvature radius in the xy-plane (e.g., a cutting plane) whereas the cutting edge of the second cutting member has portions each with a curvature radius that is chosen so as to maintain a constant cutting angle between the respective cutting edges of the first and second cutting members. For example, the cutting edge of the second cutting member comprises at least two portions, e.g., a proximal portion and a distal portion, each having a determined radius of curvature. The number of portions may be chosen so as to maintain the cutting angle as constant or substantially constant when the first and second cutting members are rotated toward one another. In some embodiments, the number of portions can be between 1 and 10 portions, including all values and sub-ranges therebetween, including, for example, 5 portions. Thus, a cutting angle between the cutting edges of each of the first and second cutting members may remain constant as the free distal ends of the first and second cutting members may be rotated toward one another to form an incision in a cutting plane. Each portion can have a determined curvature radius. The cutting angle may be between about 1 degree to about 5 degrees, including all values and sub-ranges therebetween, including, for example, between about 1.5 degrees to about 2.5 degrees, or about 1.9 degrees. Having the cutting angle remain within this range can be desirable to avoid having the effort to move the cutting members (or resistance between the cutting members) be too high or too low. The effort or resistance between the cutting members may depend on the cutting angle. Having a cutting angle that is too great (e.g., greater than about 2.5 degrees) can induce an important effort (or require a high effort), which may result in blocking or jamming the cutting members. Alternatively, if the cutting angle becomes too small (e.g., less than about 1.5 degrees), the effort may not be important enough (or sufficient enough) to maintain the material (e.g., tissue) between the cutting blades so that the material is not cut but distorted. The constant cutting angle can provide effective and substantially even cuts during use of the surgical scissors.

According to embodiments, the surgical scissors described herein can provide a constant or substantially constant cutting angle, a constant or substantially constant shear angle and a constant or substantially constant opening angle, as described below with reference to FIGS. 14A-14C.

According to some embodiments, surgical scissors having scissor blades with an integrated spring at the root of the scissor blades for use in robotic and/or laparoscopic surgery are provided. For example, the integrated spring may have a waveform or U-shape, to thereby provide a more consistent reaction force between the pair of scissor blades as the sprung surgical scissor is actuated, which results in a more effective and reliable surgical scissor. Accordingly, the sprung surgical scissors described herein do not require separate additional Belleville springs, and thus, have a reduced number of components compared to other robotic surgical scissors. Unlike other surgical scissors that integrate a spring as part of the scissor blade, the sprung surgical scissors described herein allow for articulation of the scissors as there are no components extending proximally away from the pivot point of the sprung surgical scissors.

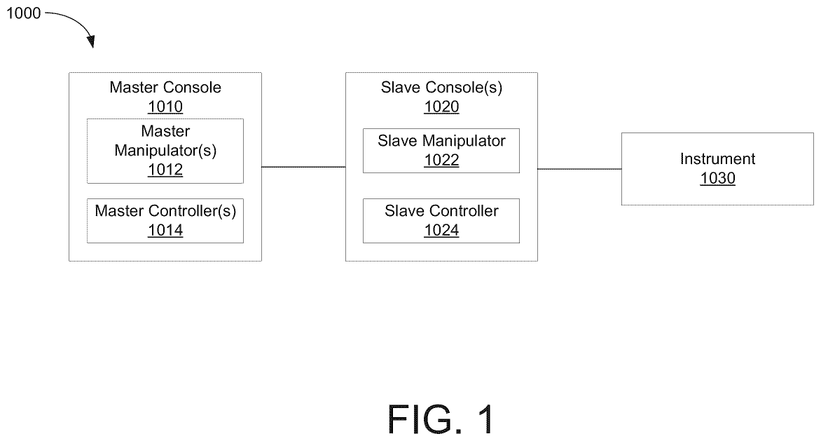

FIG. 1 schematically depicts a surgical robotic system 1000, according to embodiments. The system 1000 can include a master console 1010 and one or more slave console(s) 1020. The system 1000 can also include an instrument 1030. The master console 1010 can be operatively coupled to the slave console(s) 1020. For example, the master console 110 can be coupled to the slave console(s) 1020 via wired and/or wireless connections. The master console 1010 can include one or more master manipulator(s) 1012 and one or more master controller(s) 1014. The master manipulator(s) 1012 can include a plurality of master links that are interconnected by a plurality of joints. Movement can be applied to the master manipulator(s) 1012 by a sterile handle, which can be actuated by a sterile user (e.g., a surgeon). The movement of the master manipulator(s) 1012 and one or more actuators of the handle can be sensed, e.g., using a plurality of sensors, and transmitted to the master controller(s) 1014. In operation, the master controller(s) 1014 can send instructions to one or more slave console(s) 1020 to cause one or more drive units and/or actuators at the slave console(s) 1020 to move based on the movements applied at the master console 1010.

Each slave console 1020 can include a slave manipulator 1022 and/or an instrument 1030 that is coupled to the slave manipulator 1022. The slave manipulator 1022 can include a plurality of links that are interconnected by a plurality of joints, and the instrument 1030 can include one or more components that can be actuated in a plurality of degrees of freedom (DOFs). The slave console(s) 1020 can include one or more drive units and/or actuators that control movement of the plurality of links and joints of the slave manipulator 1022 and the component(s) of the instrument 1030. In accordance with aspects of the present disclosures, the slave manipulator 1012 and the instrument 1030 of the slave console(s) 1020 can be configured to move in a manner responsive to movements applied at the handle of the master console 1010, such that the slave manipulator 1022 and the instrument 1030 reproduces the movement applied at the handle of the master console 1010. In particular, the master console 1010 can generate instructions or commands based on movements applied at the handle and transmit those instructions or commands to the slave console(s) 1020 to cause movement of the slave manipulator 1012 and/or the instrument 1030. The slave console(s) 120 can include a slave controller 1024 that can be configured to interpret the instructions or other signals from the master console 1010 and to control the movement of the slave manipulator 1012 and/or the instrument 1030.

While the slave console 1020 is described as having a slave manipulator 1022 and an instrument 1030, it can be appreciated that a single slave console 1020 can include more than one slave manipulator 1022 and/or more than one instrument 1030. For example, a slave console 1020 can include two slave manipulators 1022 that each support one or more instruments 1030.

The master controller(s) 1014 and/or the slave controller(s) 1024, as described herein, can include one or more of a memory, a processor, a communications interface, and/or an input/output device. The memory can include any type of suitable non-transitory compute readable media that can store instructions that can be executed by one or more processors. The memory can be, for example, a random access memory (RAM), a memory buffer, a hard drive, a database, an erasable programmable read-only memory (EPROM), an electrically erasable read-only memory (EEPROM), a read-only memory (ROM), and/or so forth. The processor can be any suitable processing device configured to run and/or execute functions associated with the surgical robotic system 100. The processor can be a general purpose processor, a Field Programmable Gate Array (FPGA), an Application Specific Integrated Circuit (ASIC), a Digital Signal Processor (DSP), and/or the like. The communications interface can include wired and/or wireless interfaces for receiving information and/or sending information to other devices. The input/output device can include one or more displays, audio devices, touchscreens, keyboards, or other input or output devices for presenting information to and/or receiving information from a user.

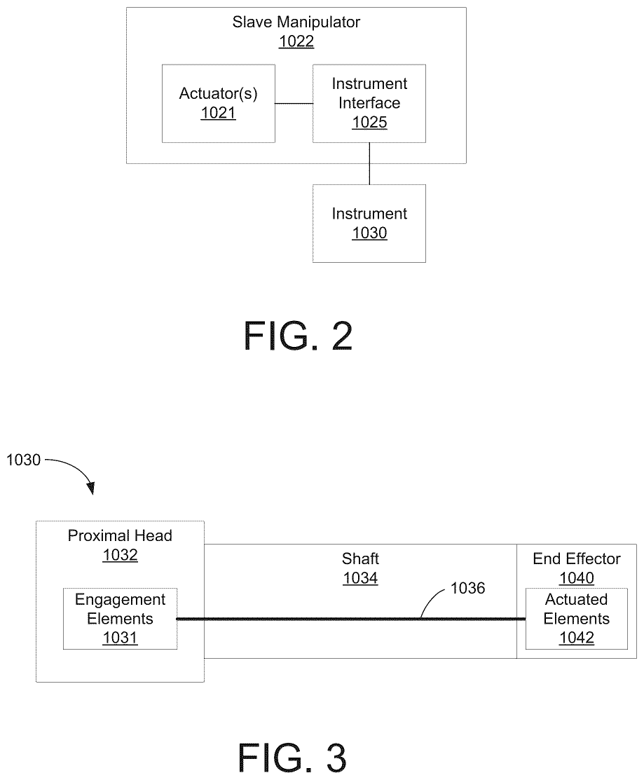

FIG. 2 schematically depicts a slave manipulator 1022 of a slave console 1020, according to embodiments. The slave manipulator 1022 can include an actuator 1021 and an instrument interface 1025. The actuator(s) 1021 can include one or more electric actuators (e.g., motors), mechanical actuators (e.g., pulleys, chains, gears, shafts, etc.), or other drive mechanisms that are configured to actuate or move one or more components of the slave manipulator 1022 and/or other components connected thereto. For example, the actuator(s) 1021 can be configured to move the plurality of links and joints of the slave manipulator 1022, the instrument interface 1025, and/or one or more component(s) of the instrument 1030. The instrument 1030 can be coupled to the actuator 1021 via the instrument interface 1025. In some embodiments, the instrument interface 1025 can include a hub for receiving the instrument 1030. The hub can be mounted on the distal end of the slave manipulator 1022, and define an opening for receiving the instrument 1030. In some embodiments, the instrument interface 1025 can include or be coupled to a sterile adapter or shield. The sterile adapter can be configured to be received within the hub, and can define a lumen for receiving a sterile instrument 1030. Suitable examples of instrument hubs and sterile shields are described with reference to International Patent Application Pub. No. WO 2018/207136, published on Nov. 15, 2018, and incorporated herein by reference. The instrument 1030, when coupled to the instrument interface 1025, can be moved by one or more actuator(s) 1021, e.g., in one or more degrees of freedom. In some embodiments, the instrument interface 1025 may be configured to receive more than one instrument 1030.

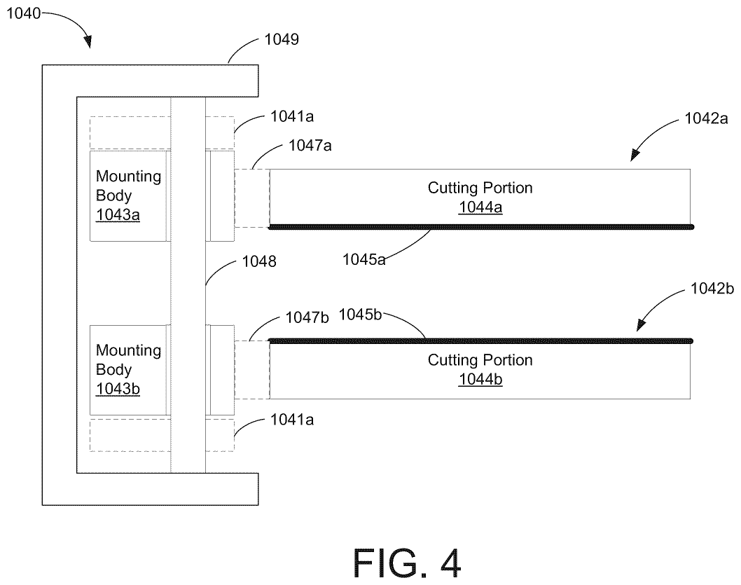

FIG. 3 schematically depicts an instrument 1030, according to embodiments. The instrument 1030 can include a proximal head 1032, a shaft 1034, and a distal end effector 1040. The proximal head 1032 can be configured to couple to the instrument interface 1025, as shown in FIG. 2. The proximal head 1032 can include one or more engagement elements or engagers 1031. The engagement elements 1031 can be coupled to one or more transmission members 1036 (e.g., force transmitting elements such as cables, wires, pulleys, rods, etc., or electrical transmitting elements such as wires, leads, electrodes, etc.) disposed in the shaft 1034 of instrument 1030. The shaft 1034 can be an elongate structure, e.g., an elongate cylinder. The shaft 1034 can define a lumen (or plurality of lumens) for housing the transmission member 1036.

In embodiments, the engagement elements 1031 include one or more extensions, protrusions, latches, tabs, hooks, ports, electrical contacts, or other suitable structure that can be configured to engage with corresponding structure of the instrument interface 1025. In an embodiment, the engagement elements 1031 can include radially extending tabs that are configured to be received in receptacles disposed in a hub of the slave manipulator 1022. The receptacles can be driven by the actuator(s) 1021 to move, to thereby transmit forces to the engagement elements 1031. Examples of suitable engagement elements (or engagers) and receptacles are described in International Patent Application Pub. No. WO 2018/207136, incorporated above by reference. While engagement elements and receptacles are described with reference to FIG. 3, it can be appreciated that any suitable form of coupling that allows the actuator(s) 1021 of the slave manipulator to couple to one or more actuated elements 1042 of the end effector 1040 to thereby actuate the actuated elements 1042 in one or more degrees of freedom can be used. For example, in some embodiments, the coupling between the instrument interface 1025 and the instrument 1030 can include a mechanical coupling (e.g., latches, pin and hole, grippers, fasteners, etc.), a magnetic coupling (e.g., electromagnets, permanent magnets, etc.), and/or an electrical coupling.

The end effector 1040 can be a surgical tool, such as, for example, a set of jaws, a clamp, a grasper, a blade, a scissor, a hook, a needle, a stapler, an electro-cautery device, an endoscope, etc. The end effector 1040 can include one or more actuated elements 1042, e.g., one, two, three, four, five, six, seven, eight, or more actuated elements. The actuated elements 1042 can be configured to be actuated (e.g., driven to move or otherwise operate) by the actuator(s) 1021 via the engagement element 1031 and the transmission elements 1036. For example, the actuated elements 1042 can include jaws, clamps, or cutting elements that can be actuated in one or more degrees of freedom, e.g., open/close, pitch, yaw, translation, etc.

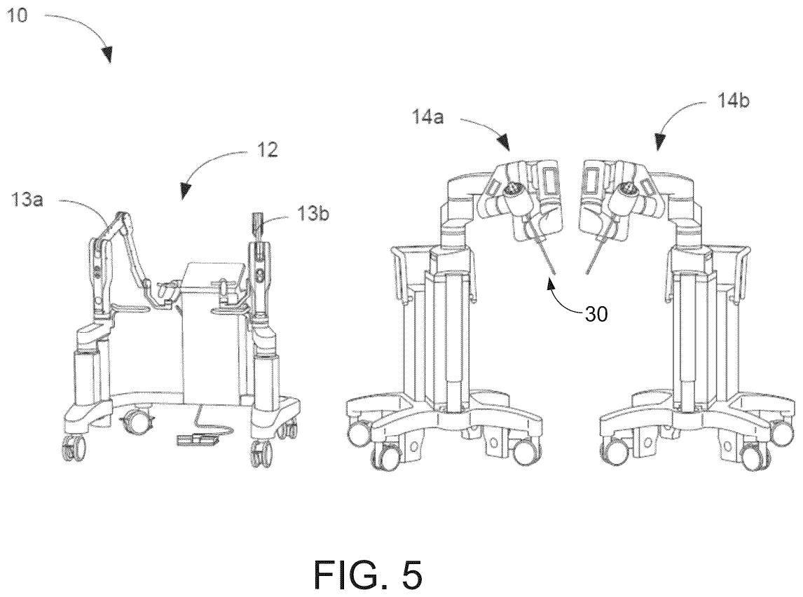

In an embodiment, the end effector 1040 can be a surgical scissor that includes a pair of cutting members or scissor blades. Accordingly, the one or more actuated elements 1042 may move (e.g., rotate, pivot, translate) in one or more degrees of freedom. In embodiments with a plurality of actuated elements (e.g., two actuated elements), the movement of the actuated elements relative to each other may facilitate opening and/or closing the end effector 1040. For example, a first actuated element may be moved (e.g., rotated) in a direction towards a second actuated element, such that cutting portions of the actuated elements may come into contact. According to some embodiments, each of the actuated elements can move toward or away from each other. In yet further embodiments, each of the actuated elements may be moved together in the same direction, such that the actuated elements may maintain an opening angle defined therebetween. The direction and magnitude of movement of the end effector 1040 can be controlled via forces applied to the engagement elements 1031 by one or more actuator(s) 1021. The movement of the end effector 1040 can provide adjustability and flexibility to the user while performing a cutting process. Further details of an instrument 1030 implemented as a surgical scissor are provided below with reference to FIG. 4.

As described above, in some embodiments, a sterile adapter may be used to facilitate the coupling between the instrument interface 1025 and the instrument 1030. The sterile adapter may be configured to maintain a sterility of an instrument 1030, while allowing for transmission of forces and/or other signals from the slave manipulator 1022 to the instrument 1030, e.g., to actuate one or more elements of the end effector 1040. The sterile adapter may include one or more mechanical and/or electrical connectors that are configured to transfer forces, energy, etc. generated at the slave manipulator 1022 to the instrument 1030. For example, the sterile adapter can include one or more movable components (e.g., sliders, cams, etc.) that allow for transmission of mechanical forces generated by the actuator(s) 1021 of the slave manipulator 1022 to the engagement element 1031 of the instrument 1030.

In operation, the instrument 1030 can be coupled to the instrument interface 1025, and a user via a user interface (e.g., at a master console 1010) can be configured to cause the actuator(s) of the slave manipulator 1022 to generate forces and/or other signals that can be transmitted via the instrument interface 1025 to the instrument 1030. In some embodiments, the forces and/or other signals can be configured to cause the actuated element(s) 1042 to move in one or more degrees of freedom, to apply energy, and/or to perform other operations.

In some embodiments, the end effector 1040 can have a pivot joint that allows for movement of one or more actuated elements 1042 in multiple degrees of freedom. FIG. 4 schematically depicts an end effector implemented as a surgical scissor 1040, according to embodiments. The end effector 1040 can include a proximal support or body 1049, a pin 1048, and actuated elements 1042a, 1042b. The actuated elements 1042a, 1042b can be cutting members, where a first cutting member includes a first mounting body 1043a and a first cutting portion 1044a, and a second cutting member includes a second mounting body 1043b and a second cutting portion 1044b. In some embodiments, the first mounting body 1043a and the first cutting portion 1044a can be monolithically formed or formed as a unitary piece, while in other embodiments, the first mounting body 1043a and the first cutting portion 1044a can be two separate structures that are coupled to one another. Similarly, the second mounting body 1043b and the second cutting portion 1044b can be formed as a unitary piece and/or formed as two separate components that are coupled to one another. As described below, the actuated elements or cutting members 1042a, 1042b can be configured to move in multiple degrees of freedom.

The proximal body 1049 can be configured to support the pin 1048. In an embodiment, the pin 1048 can be a cylindrical pin. The first and second ends of the pin 1048 can be disposed in openings defined by respective first and second portions of the body 1049. According to embodiments, a first end of the pin 1048 can be coupled to a first side of the body 1049 and a second end of the pin 1048 can be coupled to a second side of the body 1049. The body 1049 can be a fastening device, such as, for example, a clevis fastener that is configured to hold the pin 1048 in place. In some embodiments, the body 1049 can form part of a wrist or other joint of the end effector 1040. For example, the body 1049 can be the distal link of a wrist of the end effector 1040, and be coupled to a proximal link of the wrist. The distal and proximal links of the wrist can be configured to allow the end effector 1040 to move in one or more degrees of freedom (e.g., pitch, yaw, etc.).

The cutting members 1042a, 1042b can be rotatably mounted on the pin 1048 via the first and second mounting bodies 1043a, 1043b, respectively. The mounting bodies 1043a, 1043b of the cutting members 1042a, 1042b can include an opening or passage through which the pin 1048 can extend. When mounted to the pin 1048, the cutting members 1042a, 1042b can be configured to rotate about an axis of the pin 1048. Rotation of the cutting members 1042a, 1042b about the pin 1048 can cause the cutting members 1042a, 1042b to open and/or close and/or to pivot together about the axis of the pin 1048. The cutting members 1042a, 1042b include cutting portions 1044a, 1044b with cutting edges 1045a, 1045b, which can close to apply a shearing or cutting force to a material (e.g., tissue) positioned between the cutting edges 1045a, 1045b.

In embodiments, the cutting members 1042a, 1042b can be independently actuated to rotate about the pin 1048. For example, the cutting members 1042a, 1042b can each rotate about the pin 1048 in either a clockwise or counterclockwise direction. The cutting members 1042a, 1042b can be made to rotate in the same direction or in different directions. Rotating one or more of the cutting members 1042a, 1042b toward the other can bring the cutting edges 1045a, 1045b toward one another or close the two cutting edges 1045a, 1045b. Stated differently, rotating one or more of the cutting members 1042a, 1042b can cause a distal end of at least one of the cutting edges 1045a, 1045b to move toward the other and cause the contact point between the cutting edges 1045a, 1045b to advance distally. For example, rotating the cutting member 1042a clockwise and rotating the cutting member 1042b counterclockwise can bring the cutting edge 1045a towards the cutting edge 1045b to close the cutting edges 1045a, 1045b. In another example, rotating the cutting member 1042a clockwise while maintaining the cutting member 1042b in place can bring the cutting edge 1045a towards the cutting edge 1045b to close the cutting edges 1045a, 1045b (or vice versa). The rotation of the cutting members 1042a, 1042b can be controlled by forces generated by the actuator(s) 1021 and imparted to the engagement elements 1031 of the instrument 1030, as described above in reference to FIGS. 2 and 3.

According to embodiments, one or more spring elements or features can be configured to apply a reaction force (e.g., spring force) to the cutting members 1042a, 1042b. In some embodiments, springs 1041a, 1042b can be disposed between the sides of the body 1049 and the respective mounting bodies 1043a, 1043b of the cutting members 1042a, 1042b. The springs 1041a, 1041b can be configured to apply a reaction force to the respective cutting members 1042a, 1042b. The reaction force may advantageously provide a consistent reaction force between the cutting members 1042a, 1042b during rotation thereof. The consistent reaction force may be configured to press the cutting members 1042a, 1042b towards one another such that more effective and reliable cuts may be performed by the cutting edges of the cutting members 1042a, 1042b. That is, pressing the cutting members 1042a, 1042b towards one another may maintain an optimal alignment of the cutting members 1042a, 1042b during rotation of one or more of the cutting members 1042a, 1042b.

According to some embodiments, the springs 1041a, 1041b can be positioned between the sides of the body 1049 and the respective mounting bodies 1043a, 1043b. Each spring 1041a, 1401b can include an opening for receiving the pin 1048. As shown, the spring 1041a can be positioned between a first side of the body 1049 and the mounting body 1043a, and the spring 1041b can be positioned between a second side of the body 1049 and the mounting body 1043b. The springs 1041a, 1041b can be configured to apply a reaction force to the respective mounting bodies 1043a, 1043b (and therefore the respective cutting members 1042a, 1042b) when the cutting members 1042a, 1042b are being actuated to perform a cut. The reaction force can be configured to press the mounting bodies 1043a, 1043b towards one another to constrain them together. According to embodiments, each of the springs 1041a, 1041b can comprise a Belleville spring.

Additionally, or alternatively, one or more integrally formed springs 1047a, 1047b may provide a reaction force to the respective cutting members 1042a, 1042b. For example, instead of or in addition to having springs 1041a, 1041b that are separate components and configured to apply an external reaction force to the cutting members 1042a, 1042b, one or both of the cutting members 1042a, 1042b may have an integrally formed spring 1047a, 1047b. The integrally formed springs 1047a, 1047b can be disposed between the mounting bodies 1043a, 1043b and the respective cutting portions 1044a, 1044b of the cutting members 1042a, 1042b. In some embodiments, the integrally formed springs 1047a, 1047b can include one or more curvatures configured to apply a reaction force to the cutting portions 1044a, 1044b, respectively. For example, the integrally formed springs 1047a, 1047b may have a wave-like shape or one or more curvatures, such as, for example, a U-shape, a S-shape, a sinusoidal shape, a V-shape, etc. The wave-like shape of the integrally formed springs 1047a, 1047b may allow for a certain degree of lateral bending or flexibility of the cutting portions 1044a, 1044b, without increasing the overall length of the cutting members 1042a, 1042b. As will be understood by a person having ordinary skill in the art, while a wave-like shape is described for the integrally formed springs 1047a, 1047b, the integrally formed springs 1047a, 1047b may have other shaped profiles or material properties to provide a reaction force to the cutting members 1042a, 1042b.

In embodiments, the cutting portions 1044a, 1044b can be configured to move in multiple degrees of freedom. As described above, each cutting member 1042a, 1042b can be configured to rotate about the axis of the pin 1048 (also referred to as a rotation axis). The springs 1041a, 1041b also allow the cutting members 1042a, 1042b translate along the rotation axis, e.g., to compress the cutting edges 1045a, 1045b of the cutting members 1042a, 1042b against each other. In some embodiments, the mounting bodies 1043a, 1043b of the cutting members 1042a, 1042b can also define openings that allow the cutting members 1042a, 1042b to move in one or more additional degrees of freedom. For example, the mounting bodies 1042a, 1042b can define an opening that is larger than the diameter of the pin 1048 in at least one dimension, such that each mounting body 1042a, 1042b can pivot or rotate about an axis that is perpendicular or normal to the actuation direction or cutting rotation of the cutting members 1042a, 1042b. These additional degrees of freedom can allow for one unique contacting point during actuation of the cutting members 1042a, 1042b. Further details of the movements of the actuated elements or cutting members of an end effector are provided below with reference to FIGS. 14A-16.

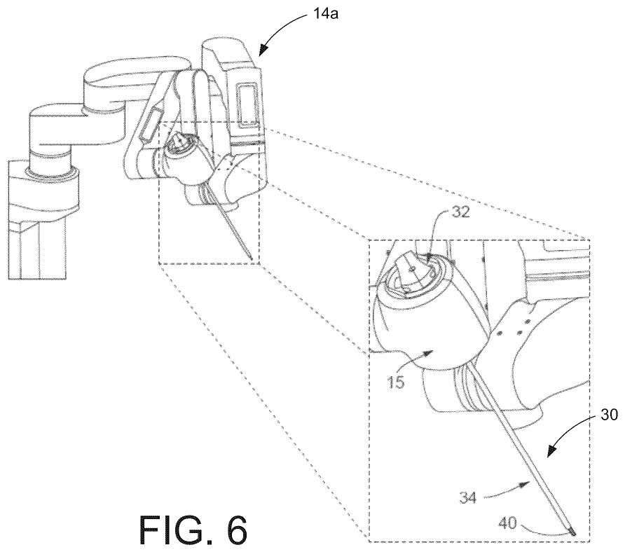

FIG. 5 depicts a surgical robotic system, according to embodiments. As shown, the surgical robotic system 10 includes a master console 12, which can be coupled to one or more slave consoles 14a, 14b. The master console 12 and the slave consoles 14a, 14b can be structurally and/or functionally similar to other master consoles and slave consoles described herein, respectively, including, for example, master console 1010 and slave console(s) 1020. Suitable examples of master consoles and slave consoles are also described in International Patent Application Pub. No. WO 2019/155383, published on Aug. 15, 2019, and International Patent Application Pub. No. WO 2020/141487, published on Jul. 9, 2020, the disclosures of both of with are incorporated herein by reference.

As shown in FIG. 5, instrument(s) 30 may be used with a teleoperated robotic surgical system 10. Each slave console 14a, 14b can include a slave manipulator that can receive an instrument 30, similar to that described with reference to FIGS. 2 and 3. The master console 12 can be operatively coupled to the slave console(s) 14a, 14b via a wired connection (e.g., electrical cables) and/or wireless connection. The master console 12 can include one or more master manipulators 13a, 13b, which can be actuated by a user (e.g., a surgeon) to cause one or more actuators (e.g., motors) of a slave console 14a, 14b to apply movements to an end effector of the instrument 30. Preferably, the slave manipulator(s) of the slave console(s) 14a, 14b (or links and joints thereof) are configured to move in a manner such that the end effector replicates the movement applied at the handle of master console 12, without deviating, during operation of surgical robot system 10. Thus, translation degrees of freedom (e.g., left/right, upward/downward, inward/outward), articulation degrees of freedom (e.g., pitch, yaw, open/close, and rotation such as pronosupination), etc. are electromechanically replicated via sensors, actuators, and a controller (e.g., slave controller 1024) of the slave console(s) 14a, 14b.

Master console 12 may be positioned within an operating room where a user (e.g., surgeon) may be situated, and in close proximity to slave console(s) 14a, 14b where a patient undergoing surgery may be situated, so that the user may move quickly between master console 12 and slave console(s) 14a, 14b, e.g., to manually perform laparoscopy during the surgery if necessary. In some embodiments, master console 12 may be covered with a sterile drape, and may include removable, sterile handles that a surgeon can manipulate, e.g., to actuate the end effector of an instrument 30. The master console 12 may include a left master manipulator 13a and a right master manipulator 13b. Left master manipulator 13a and right master manipulator 13b and may be positioned on a single master console 12 such that left master manipulator 13a may be manipulated by the surgeon's left hand and right master manipulator 13b may be manipulated by the surgeon's right hand, when the surgeon is situated at master console 12. Left master manipulator 13a and right master manipulator 13b may be operated simultaneously and/or independently from the other, e.g., by the surgeon's right and left hands.

As further illustrated in FIG. 5, the slave console(s) 14a, 14b may comprise a left slave manipulator operatively coupled to the left master manipulator 13a, and a right slave manipulator operatively coupled to right master manipulator 13b. The slave manipulators may be positioned on separate consoles, where one slave console may be positioned on a first side of the patient undergoing surgery, and another slave console may be positioned on the first side or another side of the patient undergoing surgery.

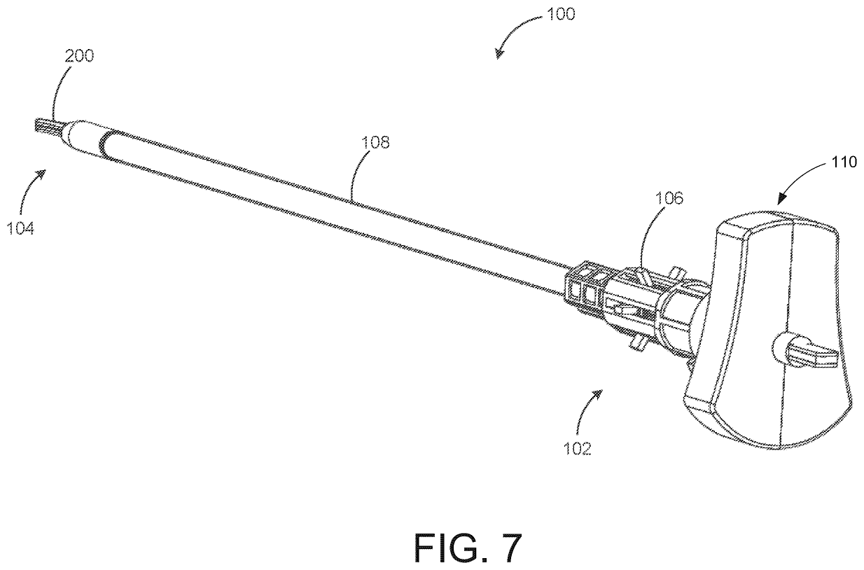

FIG. 6 provides a close-up view of an instrument 30 positioned in a hub of a slave manipulator of a slave console 14a, according to embodiments. As shown in FIG. 6, the instrument 30 has a proximal head 32, a shaft 34, and a distal end effector 40. The instrument 30 can be structurally and/or functionally similar to other instruments described herein, including for example, instrument 1030. The proximal head 32 of the instrument 30 can be releasably coupled to a hub 15 of the slave manipulator. The hub 15 can define an opening through which the instrument 30 can be inserted. The instrument 30, after being inserted into the hub 15 and coupled to the slave manipulator, can be configured to be actuated in one or more degrees of freedom, as described above.

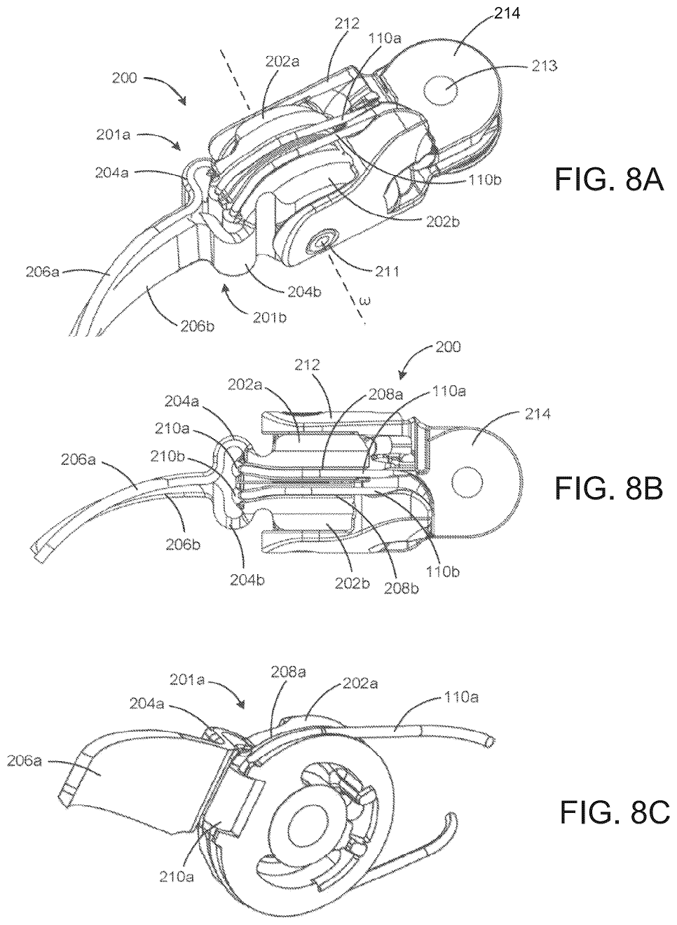

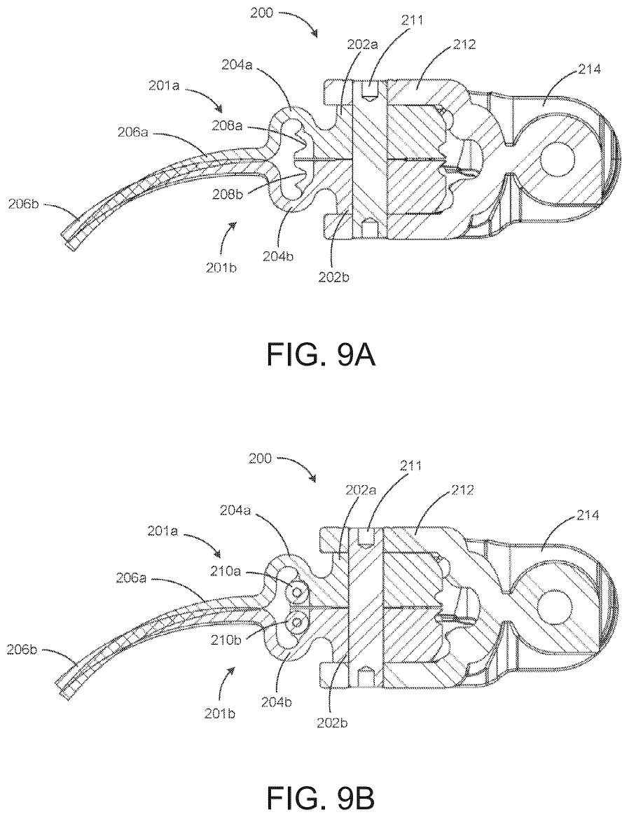

Referring now to FIG. 7, an example surgical instrument is provided. The surgical instrument 100 can be structurally and/or functionally similar to other instruments described herein, including, for example instruments 1030, 30. Surgical instrument 100 may include a proximal region 102 including an instrument hub or head 110, a distal region 104 having an end effector 200, and an instrument shaft 108 extending between the proximal region 102 and the distal region 104. The end effector 200 can be implemented as a surgical scissor, as shown in greater detail in FIGS. 8A-10C. In some embodiments, the end effector 200 may be removable from the shaft 108. In some embodiments, the shaft 108 may be removable from the head 110 and/or end effector 200.

As shown in FIG. 7, the instrument 100 may include one or more pairs of engagers 106 configured to be actuated to actuate end effector 200 in one or more degrees of freedom, e.g., pitch, yaw, and open/close. For example, engagers 106 may be operatively coupled to end effector 200 via a plurality of force transmitting elements, e.g., cables, extending from engagers 106 through instrument shaft 108 to end effector 200. As described in further detail below, a first pair of engagers of engagers 106 may be actuated to actuate end effector 200 in a yaw degree of freedom. A second pair of the engagers of engagers 106 may be operatively coupled to a first scissor blade of end effector 200 and configured to actuate movement of the first scissor blade, and a third pair of engagers of engagers 106 may be operatively coupled to a second scissor blade of end effector 200 and configured to actuate movement of the second scissor blade. Actuation of the first and second scissor blades in the same direction can actuate end effector 200 in a pitch degree of freedom, and actuation of the first and second scissor blades in opposite directions actuates end-effector 200 in an open/close degree of freedom. The one or more pairs of engagers 106 may be removably engaged with corresponding structures of a hub of a slave console, e.g., via a releasable hook mechanism, such that movements at a handle of a master console (e.g., operated by a surgeon) may be replicated at end effector 200 of surgical instrument 100.

Referring now to FIGS. 8A to 8C, more detailed views of the surgical instrument 200 are provided. As shown, in some embodiments, the surgical instrument 200 can be a sprung surgical scissor for use in robotic and/or laparoscopic surgery. Sprung surgical scissor 200 may include upper scissor portion (or a first scissor portion) 201a rotatably coupled to lower scissor portion (or a second scissor portion) 201b, e.g., about axis @. While upper and lower are used to refer to the scissor portions 201a, 201b throughout the paragraphs that follow, it can be appreciated that upper and lower are not intended to impart any specific arrangement of the scissor portions 201a, 201b other than to indicate that there are two separate scissor portions 201a, 201b that are configured to interact with one another.

In some embodiments, upper scissor portion 201a and lower scissor portion 201b may be formed via, metal injection molding (MIM), metal three-dimensional (3D) printing, and/or by milling. Upper scissor portion 201a may include mounting body 202a and cutting portion 206a (e.g., a curved blade), which extends distally from mounting body 202a. At a proximal end or root of the cutting portion 206a, e.g., the end of cutting portion 206a closer to mounting portion 202a, a spring 204a can be integrally formed with cutting portion 206a and/or mounting body 202a. In some embodiments, cutting portion 206a and spring 204a are integrally formed with mounting body 202a. Accordingly, mounting body 202a narrows and becomes spring 204a, which goes directly into the blade of cutting portion 206a. In some embodiments, cutting portion 206a itself may have a spring force. Moreover, cutting portion 206a may have a sharp cutting inner edge and a blunt outer edge.

As shown in FIGS. 8A to 8C, spring 204a may have a wave-like shape, e.g., a U-shape. For example, spring 204a may extend upwardly and distally from mounting portion 202a toward an apex, and then downwardly and distally from the apex to the cutting portion 206a. Cutting portion 206a may then extend distally from spring 204a with a slight, predefined curve, e.g., in a downward direction. The spring 204a therefore provides a region having a longer chord length, thereby providing cutting portion 206a flexibility without increasing (or without significantly increasing) the overall length of the blade. The spring 204a also allows bending stresses to be distributed along a length of cutting portion 206a, instead of being concentrated at a root or base of the cutting portion 206a. As will be understood by a person having ordinary skill in the art, spring 204a may have other shaped profiles to thereby provide a consistent reaction force of upper scissor portion 201a in the downward direction, e.g., against lower scissor portion 201b. For example, spring 204a may have a sinusoid shape, a V-shape, etc.

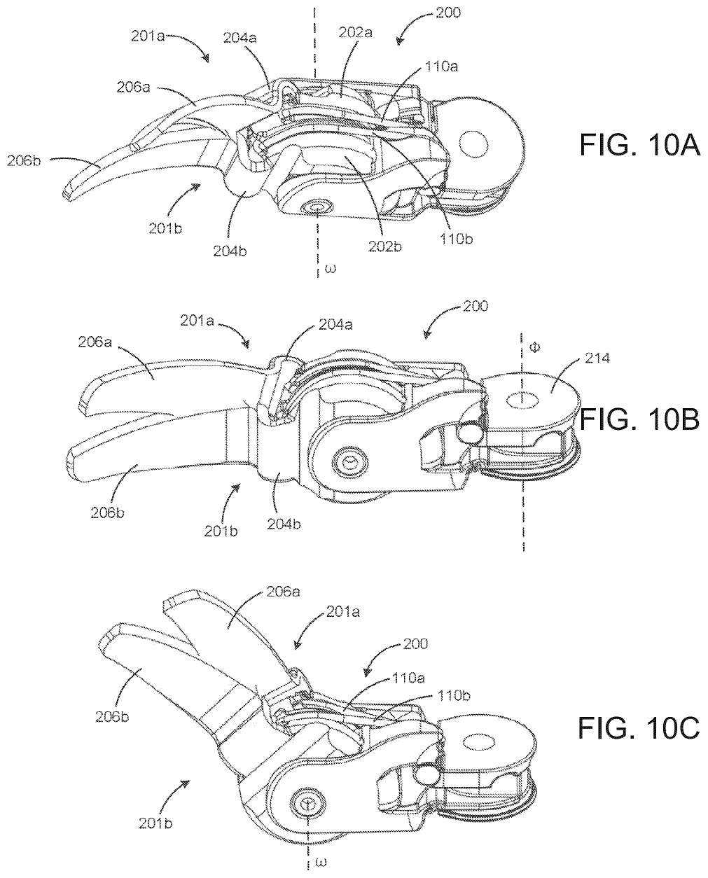

In addition, mounting body 202a may have a circular profile, and may have groove 208a at least partially extending circumferentially along an outer edge of mounting body 202a, as shown in FIG. 9A. Groove 208a may be sized and shaped to receive one or more force transmitting elements, e.g., cable 110a. Cable 110a coupled to mounting body 202a may be coupled to an engager (e.g., engager 106) at a proximal end of the instrument, such that actuation of the engager causes actuation of cable 110a, which causes rotation of mounting body 202a, and accordingly upper scissor portion 201a. For example, cable 110a may be a single cable that has one end coupled to a first engager of a pair of engager, and loops around groove 208a of mounting body 202a, such that the other end of cable 110a is coupled to a second engager of the pair of engagers. Accordingly, the pair of engagers may be actuated in equal and opposite directions to thereby cause rotation of mounting body 202a via cable 110a. Alternatively, cable 110a may include two separate cables, each coupled to a respective engager of the pair of engagers at one end, and to mounting body 202a at the other end. Moreover, mounting body 202a may include a crimp 210a configured to secure cable 110a to groove 208a, such that cable 110a is fixed to mounting body 202a, as shown in FIGS. 8C and 9B.

Lower scissor portion 201b may be constructed similar to upper scissor portion 201a. For example, as shown in FIGS. 8A and 8B, lower scissor portion 201b may include mounting body 202b and cutting portion 206b, e.g., a curved blade, extending distally from mounting body 202b. At a proximal end or root of the cutting portion 206b, e.g., the end of cutting portion 206b closer to mounting portion 202b, a spring 204b can be integrally formed with cutting portion 206b and/or mounting body 202b. In some embodiments, cutting portion 206b and spring 204b are integrally formed with mounting body 202b. Accordingly, mounting body 202b narrows and becomes spring 204b, which goes directly into the blade of cutting portion 206b. In some embodiments, cutting portion 206b itself may have a spring force. Moreover, cutting portion 206b may have a sharp cutting inner edge and a blunt outer edge, such that sharp cutting inner edge of cutting portion 206b interacts with the sharp cutting inner edge of cutting portion 206a as upper scissor portion 201b rotates relative to lower scissor portion 201a. Alternatively, only one of the inner edges of cutting portion 206a or cutting portion 206b may have a sharp cutting edge, such that the other inner edge may be blunt. As shown in FIGS. 8A and 8B, spring 204b may have a wave-like shape, e.g., a U-shape, similar to the spring 204a. While spring 204a and spring 204b are shown in FIGS. 8A-8B as having a similar shape, it can be appreciated that in some embodiments, spring 204a may have a first shape while spring 204b has a second shape that is different from the first shape.

In some embodiments, cutting portions 206a, 206b may extend distally in a non-curved manner from springs 204a, 204b, respectively. As will be understood by a person having ordinary skill in the art, cutting portions 206a, 260b may be longer or shorter than is illustrated in FIGS. 8A and 8B. In addition, in some embodiments, the spring-based mechanisms described herein may be used with passive surgical instruments (e.g., instruments without electrosurgery) or with electrosurgical instruments. Moreover, cutting portions 206a, 206b may have greater friction at certain areas or portions along cutting portions 206a, 206b, e.g., by selecting the material for cutting portions 206a, 206b to increase friction therebetween, or treating at least a portion of the surfaces of cutting portions 206a, 206b, for example, by applying lubricants, coatings, or other finishes.

In addition, mounting body 202b may have a circular profile, and may have groove 208b at least partially extending circumferentially along an outer edge of mounting body 202b, as shown in FIG. 9A. Groove 208b may be sized and shaped to receive one or more force transmitting elements, e.g., cable 110b. Cable 110b can function similarly as cable 110a, and can be configured to cause rotation of mounting body 202b, and accordingly lower scissor portion 201b. For example, cable 110b coupled to mounting body 202b may be coupled to an engager (e.g., engager 106), such that actuation of the engager causes actuation of cable 110b, which causes rotation of mounting body 202b, and accordingly lower scissor portion 201b. In some embodiments, cable 110b may be a single cable, while in other embodiments, cable 110 may be two cables. Mounting body 202b may include crimp 210b configured to secure cable 110b to groove 208b, such that cable 110b is fixed to mounting body 202b, as shown in FIG. 9B.

As shown in FIG. 8A, sprung surgical scissor 200 further may include a body or frame having distal portion 212 and proximal portion 214. The frame can be functionally and/or structurally similar to the body 1049, as described above with reference to FIG. 4. Mounting body 202a and mounting body 202b may be disposed within and rotatably coupled to distal portion 212 of the frame via pin 211, as shown in FIGS. 9A and 9B, such that distal portion 212 pushes mounting body 202a and mounting body 202b toward each other. Mounting body 202a may define an opening for receiving the pin 211 that is concentric with a similar opening of mounting body 202b, such that mounting bodies 202a, 202b are both configured to rotate relative to the frame about axis @ of sprung surgical scissor 200, which extends along the longitudinal axis of pin 211. When mounting bodies 202a, 202b are rotatably coupled together within the frame, springs 204a, 204b causes cutting portions 206a, 206b, respectively, to apply a consistent reaction force against each other.

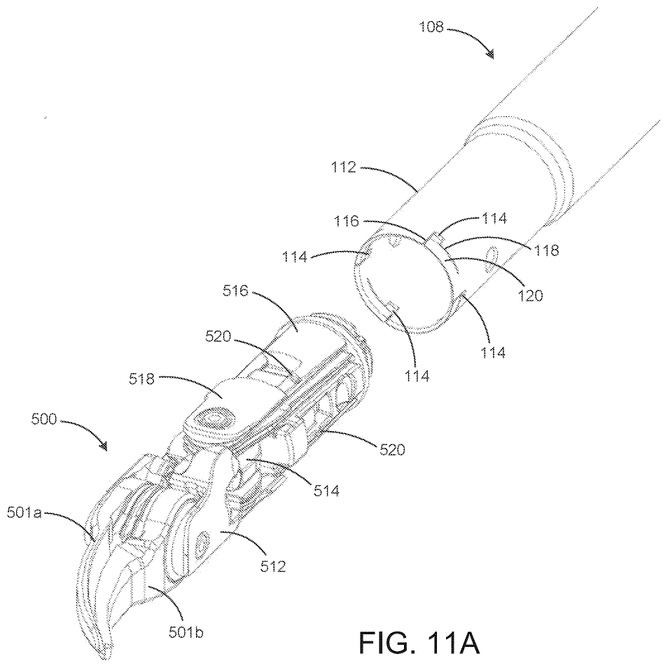

FIGS. 10A-10C illustrate example configurations of the surgical scissor 200 described herein. For example, as described above, mounting bodies 202a, 202b may be actuated to rotate in opposite directions about the axis ω via force transmitting elements 110a, 110b (e.g., cables), respectively, to actuate sprung surgical scissor 200 in the open/close degree of freedom, as shown in FIG. 10A. Mounting bodies 202a, 202b may also be actuated to rotate in the same direction to actuate sprung surgical scissor 200 in the pitch degree of freedom, as shown in FIG. 10C. As will be understood by a person having ordinary skill in the art, only one of mounting body 202a or mounting body 202b needs to be rotated relative to the other to actuate the open/close degree of freedom of sprung surgical scissor 200. Proximal portion 214 of frame may be rotatably coupled to instrument shaft 108 of surgical instrument 100. Proximal portion 214 may be coupled to one or more other force transmitting elements, such that actuation of the force transmitting elements coupled to proximal portion 214 causes the frame, and accordingly upper and lower scissor portions 201a, 201b, to rotate about pivot point 213 of proximal portion 214, e.g., about axis @ as shown in FIG. 10B. Axis @ of pivot point 213 of proximal portion 214 may be perpendicular to axis @ of distal portion 212. Accordingly, actuation of the force transmitting element coupled to proximal portion 214 may cause actuation of sprung surgical scissor 200 in the yaw degree of freedom, as shown in FIG. 10B.

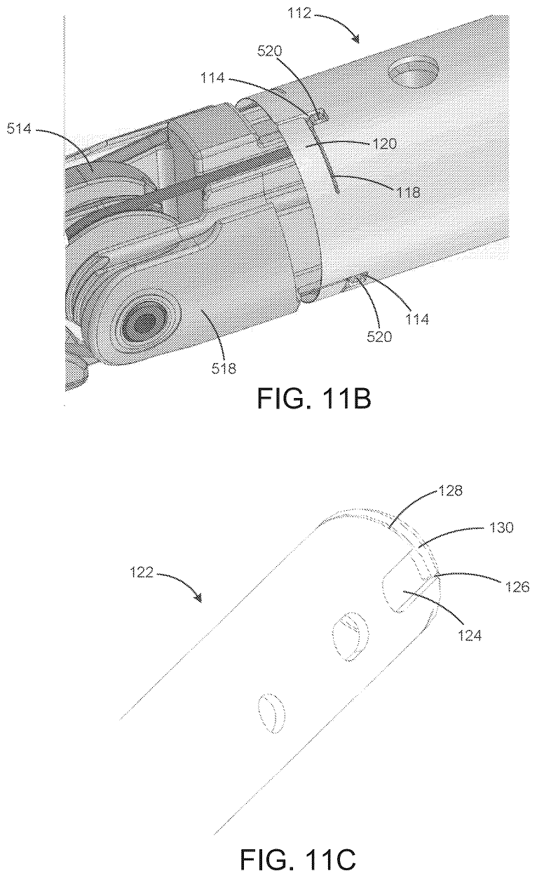

Referring now to FIGS. 11A and 11B, an example coupling mechanism for coupling an end effector to an instrument shaft is provided. Although FIG. 11A illustrates a surgical scissor end effector without an integrated spring as described above, as will be understood by a person having ordinary skill in the art, sprung surgical scissor 200 (and any other instruments as described herein) also may incorporate the coupling mechanism described herein. End effector 500 may be constructed similar to other end effectors described herein, including, for example, end effector 1040, 200. For example, as shown in FIG. 11A, end effector 500 may include upper scissor portion (or first scissor portion) 501a and lower scissor portion (or second scissor portion) 501b, which may be actuated in a similar manner as upper scissor portion 201a and lower scissor portion 201b, respectively, as described above. For example, upper and lower scissor portions 501a, 501b may be actuated to rotate via their respective mounting portions in opposite directions relative to distal frame portion 514 to actuate the open/close degree of freedom, and in the same direction to actuate the pitch degree of freedom. Moreover, end effector 500 may be actuated such that proximal frame portion 514 rotated relative to distal connection portion 518 to thereby actuate the yaw degree of freedom.

As shown in FIG. 11A, end effector 500 may include a connection portion having proximal connection portion 516 and distal connection portion 518. Proximal connection portion 516 may be sized and shaped to be received within a lumen of distal portion 112 of instrument shaft 108. As shown in FIG. 11A, proximal connection portion 516 may include one or more knobs 520 disposed about the circumference of proximal connection portion 516. For example, knobs 520 may be evenly spatially distributed about the circumference of proximal connection portion 516. Knobs 520 may have a geometry configured to facilitate advancement of proximal connection portion 516 within distal portion 112 of instrument shaft 108. For example, the proximal side of knobs 520 may be tapered/angled. Moreover, the distal side of knobs 520 may have a geometry configured to facilitate securement of knobs 520 to distal portion 112, as described in further detail below

As further shown in FIG. 11A, distal portion 112 of instrument shaft 108 may include one or more flexible flaps 120 disposed along the circumference of distal portion 112. For example, the number of flexible flaps 120 may correspond with the number of knobs 520. Moreover, there may be at least as many flexible flaps 120 as there are knobs 520. Flexible flaps 120 may be defined by lateral cut 116 extending along the longitudinal axis of instrument shaft 108, and circumferential cut 118 extending along the circumference of distal portion 112, such that flexible flaps 120 extends from a distal end of distal portion 112 towards circumferential cut 118. Accordingly, flexible flap may expand radially outward responsive to an outwardly radial force applied thereon.

In addition, distal portion 112 further may include one or more openings 114 disposed proximal to one or more flexible flaps 120. For example, each opening 114 may be disposed proximally to each flexible flap 120. Openings 114 may be formed during manufacturing along with the formation of lateral cut 116 and circumferential cut 118. Openings 114 may be sized and shaped to receive knobs 520 therein. Accordingly, as proximal connection portion 216 is advanced through the lumen of distal portion 112, knobs 520 engage with flexible flaps 120, such that the tapered proximal side of knobs 520 applies an outwardly radially force to flexible flaps 120, thereby causing flexible flaps 120 to expand radially outward as knobs 520 moves proximally relative to flexible flaps 120.

Proximal connection portion 216 may be advanced proximally relative to distal portion 112 until knobs 520 are disposed within openings 114, and flexible flaps 120 collapses back to their natural state, as shown in FIG. 11B. As described above, the distal side of knobs 520 may have a geometry to facilitate securement of knobs 520 within openings 114. For example, the distal side of knobs 520 may be flat to thereby prevent distal movement of proximal connection portion 216 from causing flexible flaps 120 to expand radially outward. Accordingly, when knobs 520 are disposed within openings 114, end effector 500 is locked to instrument shaft 108 via the flat distal side of knobs 520 and flexible flaps 120.

As shown in FIG. 11C, the proximal end of instrument shaft also may include a similar coupling mechanism for coupling to the instrument hub, as described above. For example, proximal portion 112 of instrument shaft 108 may include one or more flexible flaps 130 defined by lateral cut 126 and circumferential cut 128, such that flexible flaps 130 extend from the proximal end of proximal portion 122 towards circumferential cut 128. Accordingly, flexible flaps 130 may expand radially outward responsive to an outwardly radial force applied thereon, e.g., via corresponding knobs on the instrument hub which may be constructed similar to knobs 520. Moreover, proximal portion 122 further may include one or more openings disposed distal to flexible flaps 130. Openings 124 may be sized and shaped to receive the corresponding knobs of the instrument hub therein, such that flexible flaps 130 secures the knobs within openings 124.

Accordingly, as a portion of the instrument hub is advanced through the lumen of proximal portion 122, the knobs of the instrument hub engage with flexible flaps 130, such that the tapered distal side of the knobs applies an outwardly radial force to flexible flaps 130, thereby causing flexible flaps 130 to expand radially outward as the knobs moves distally relative to flexible flaps 130. The instrument hub may be advanced distally relative to distal proximal 122 until the knobs are disposed within openings 124, and flexible flaps 130 collapses back to their natural state, thereby securing the knobs within openings 124, and locking the instrument hub to instrument shaft 108. For example, the proximal side of the knobs of the instrument hub may be flat to facilitate securement of the knobs within openings 124.

As will be understood by a person having ordinary skill in the art, the coupling mechanism described herein may be used to couple other types of end effectors and instrument hubs to the instrument shaft, such as those described in International Patent Application Pub. No. WO 2019/155383 and International Patent Application Pub. No. WO 2020/141487, incorporated above by reference

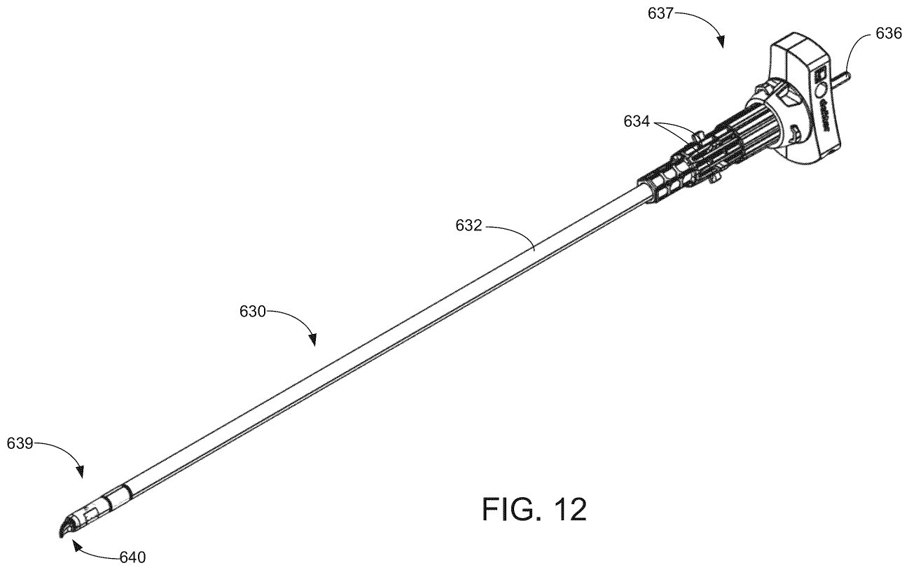

Referring now to FIG. 12, another variation of an example surgical instrument 630 is provided. The surgical instrument 630 can be functionally and/or structurally similar to other surgical instruments described herein, including, for example, surgical instruments 1030, 30, 100. For example, the surgical instrument 630 includes a proximal region 637 including a proximal head, a distal region 639 including an end effector implemented as a surgical scissor 640, and an instrument shaft 632 extending between the proximal region 637 and the distal region 639.

The surgical instrument 630 includes an electrical connector 636, which can be configured to connect to an electrical port or other electrical connector, e.g., to establish an electrical coupling and enable use of the surgical instrument 630 as an electrosurgical device (e.g., ablation device, electro-cautery device, electro-coagulation device, etc.) and/or to otherwise allow for electrically driven operation of the surgical instrument 630. Alternatively, in some embodiments, the surgical instrument 630 may be a mechanically operated surgical instrument that does not include an electrical connector 636.

The surgical instrument 630 also includes a plurality of engagers 634, which can be coupled to one or more actuators (e.g., motors) of a slave manipulator (e.g., slave manipulator 1022) to enable actuation of one or more actuated elements of the surgical scissor 640. The coupling between the plurality of engagers 634 and the actuators of the slave manipulator can be structurally and/or functionally similar to that of instrument 1030 and/or 100, described above with reference to FIGS. 2 and 7. For example, the engagers 634 may be coupled via receptacles to one or more actuator(s) of a slave manipulator, and the engagers 634 may also be coupled to force transmitting elements (e.g., cables) to surgical scissor 640. Actuation of the engagers 634, in response to forces generated by the actuator(s) of slave manipulator, can cause the engagers 634 to move (e.g., linearly translate), thereby actuating one or more elements of surgical scissor 640 in one or more degrees of freedom, e.g., pitch, yaw, and open/close. In some embodiments, the engagers 634 (and the instrument 630) are configured to releasably coupled to the slave manipulator, such that the instrument 630 can be coupled to and decoupled from the slave manipulator. While not shown or described with reference to FIG. 12 or the following figures, it can be appreciated that a sterile adapter can be used with the surgical instrument 630, thereby providing a sterile coupling between the instrument 630 and the slave manipulator. When the surgical instrument 630 is coupled to the slave manipulator, movements at a handle of a master console (e.g., operated by a surgeon) may be replicated or cause movement of one or more components of the surgical scissor 640.

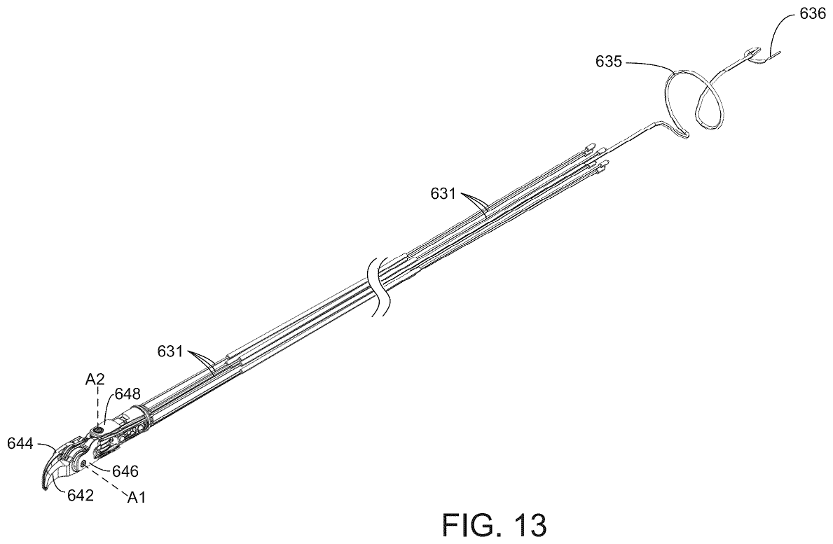

FIG. 13 shows the surgical instrument 630 of FIG. 12, without outer components of the instrument 630 being shown (e.g., shaft 632, instrument head, etc.), to aid in visualizing a plurality of force transmitting elements 631 (e.g., cables) that are disposed within the shaft 632. The force transmitting elements 631 can be structurally and/or functionally similar to the transmission members 1036, as described above with reference to FIG. 3. As shown in FIG. 13, the electrical connector 636 can be coupled to an electrical transmitting element 635 (e.g., a wire or lead). The force transmitting elements 631 each can have a proximal end that is coupled to a respective one of the engagers 634 (as shown in FIG. 12) and a distal end that is coupled to an actuated component of the surgical scissor 640. The surgical scissor can include a pair of cutting members 642, 644 that are held together by a first frame or body 646. The body 646 includes a distal portion that is configured to hold a pin 641 (depicted in later figures) in place, which in turn rotatably supports the cutting members 642, 644. As such, the distal portion of the body 646 functions similar to a clevis. The body 646 further includes a proximal portion that is configured to rotatably couple to a second frame or body 648, e.g., via a similar clevis and pin mechanism. In operation, the cutting members 642, 644 can be configured to rotate about an axis A1 of the pin 641, and the body 646 can be configured to rotate about the axis A2, as shown in FIG. 13.

The force transmitting elements 631 can include a first pair of force transmitting elements that are coupled to the body 646 and, in response to forces applied at engagers 634 coupled to the pair of force transmitting elements, can actuate the body 646 to rotate about the axis A2, e.g., in a yaw degree of freedom. The force transmitting elements 631 can also include a second pair of force transmitting elements and a third pair of force transmitting elements that can couple to the first cutting member 642 and the second cutting member 644, respectively, and in response to forces applied at engagers 634 coupled to the second and third pairs of force transmitting elements, can actuate the first and second cutting members 642, 644 to rotate about the axis A2. In embodiments, the first and second cutting members 642, 644 may rotate about the pin 641 independently. That is, the cutting member 642 may rotate in a first or second direction about the pin 641 and the second cutting member 644 may rotate in the first or a second direction about the pin 641. Actuation in the same direction of the first and second cutting members 642, 644, responsive to forces transmitted via the second and third pairs of force transmitting elements, actuates end effector 640 in a pitch degree of freedom, and actuation in opposite directions of the first and second cutting members 642, 644 (or actuation of one of the first and second cutting members 642, 644, without actuating the other) actuates end effector 640 in an open/close degree of freedom.

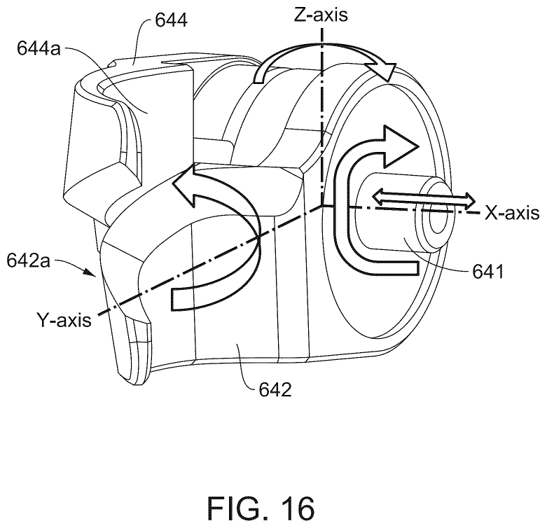

FIGS. 14A-14C illustrate motions and associated parameters of the surgical scissor 640. As depicted, the surgical scissor 640 includes a first cutting member 642, a second cutting member 644, a pin 641, and a body or frame 646 supporting the pin. The first and second cutting members 642, 644 may be coupled together by the pin 641 and the body 646. The pin 641 can define an axis (e.g., axis A2), which is similar to the axis @ described in reference to FIGS. 8A-8C. The first and second cutting members 642, 644 can engage in a cutting rotation, whereby at least one of the first and second cutting members 642, 644 rotates about the axis of the pin 641 toward the other, e.g., to perform a cut. The angle formed between the first and second cutting members 642, 644 while performing the cut can referred to as a cutting angle. The cutting angle may be defined at the point of contact of the cutting edges of the cutting members 642, 644. As shown in FIG. 14A, the cutting angle can be determined in an xy-plane of the end effector (as defined based on the axes shown in FIG. 16).

In some embodiments, surgical scissor 640 as described herein (and other surgical scissors described herein) can provide a constant or substantially constant cutting angle. The curvature of one of the cutting members, e.g., first cutting member 642, may be predetermined, whereas the curvature of the other cutting member, e.g., second cutting member 644, may be defined depending on the predetermined curvature of the first cutting member 642 so as to maintain a constant or substantially constant cutting angle between the respective cutting edges of the first and second cutting members. For example, the cutting edge of the second cutting member can include at least two portions, e.g., a proximal portion and a distal portion, each having a determined radius of curvature. The number of portions may be selected so as to maintain the cutting angle as constant or substantially constant when the first and second cutting members 642, 644 are rotated toward one another. In some embodiments, the number of portions can be between 1 and 10 portions, including all values and sub-ranges therebetween, including, for example, 5 portions. Thus, a cutting angle between the cutting edges of each of the first and second cutting members 642, 644 may remain constant as the free distal ends of the first and second cutting members may be rotated toward one another to form an incision in a cutting plane. The cutting angle may be between about 1 degree to about 5 degrees, including all values and sub-ranges therebetween, including, for example, between about 1.5 degrees to about 2.5 degrees.

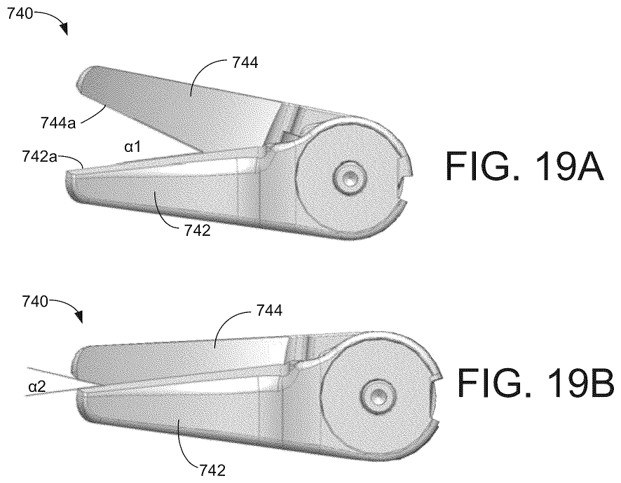

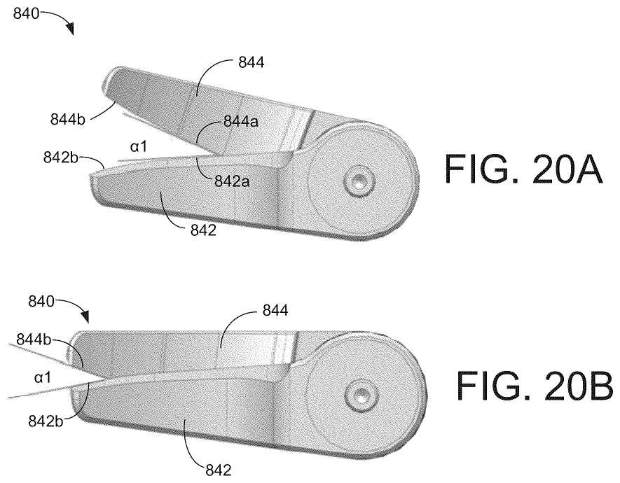

The first and second cutting members 642, 644 can engage in an opening rotation, whereby at least one of the first and second cutting members 642 rotates about the axis of the pin 641 away from the other, e.g., to open. The angle formed between the first and second cutting members 642, 644 while opening can be an opening angle. The opening angle may be defined at the point of contact of the cutting edges of the cutting members 642, 644. As shown in FIG. 14B, the opening angle can be determined in an yz-plane of the end effector (as defined based on the axes shown in FIG. 16). The opening angle can correspond to a slice-push ratio, as will be described further in reference to FIGS. 19A-19B and 20A-20B.

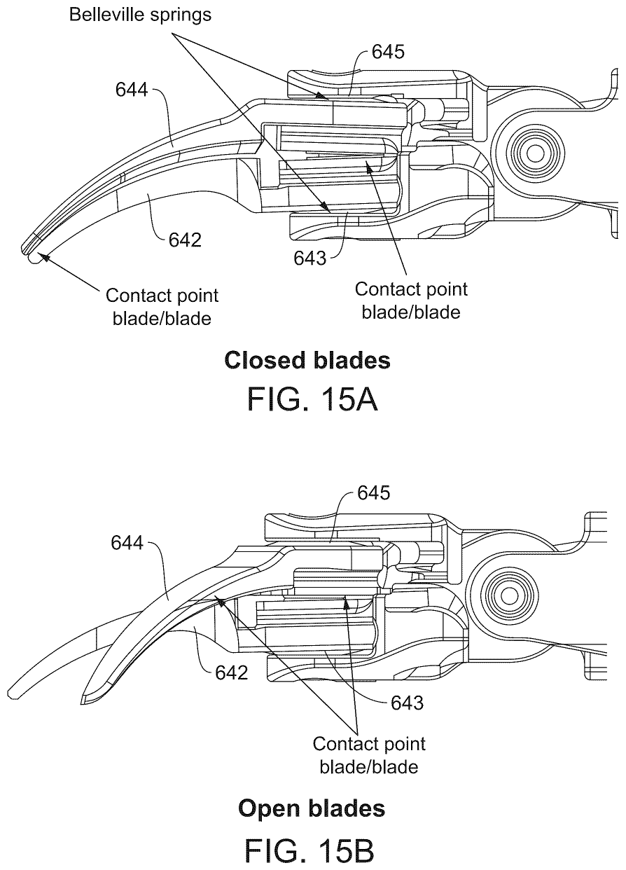

In some embodiments, the first and second cutting members 642, 644 can also be configured to translate along the axis of the pin. The translation of the cutting members 642, 644 can be controlled or limited by one or more springs (e.g., Belleville springs) that are disposed between each cutting member 642, 644 and the body 646. As shown in FIG. 15A-15B, springs 643, 645 can be configured to press the cutting members 642, 644 toward one another, thereby constraining their translation about the axis of the pin while allowing a limited amount of axial translation. In particular, the surgical scissor 640 includes a first spring 643 that is disposed between the first cutting member 642 and a first side of the body 646 and a second spring 645 that is disposed between the second cutting member 644 and a second side of the body 646. The first and second springs 643, 645 may apply an elastic force to the respective cutting members 642, 644. For example, the elastic force applied by the springs 643, 645 may push the cutting members 642, 644 toward one another. The spring force applied by the springs 643, 645 causes the cutting members 642, 644 to apply a consistent reaction force against each other as they are performing a cutting rotation. FIG. 15A depicts the cutting members 642, 644 in a closed configuration, and FIG. 15B depicts the cutting members 642, 644 in an open configuration. In both configurations, the cutting members 642, 644 are constrained together such that they contact one another at a proximal end and have one unique contact point where the respective cutting edges of the cutting members 642, 644 contact one another.

The first and second cutting members 642, 644 can also form a shear angle, as shown in FIG. 14C. The shear angle, as shown in the xz-plane of the end effector (as defined based on the axes shown in FIG. 16), can be impacted if one or both of the cutting members 642, 644 were to rotate about their axes (e.g., rotate or tilt about a longitudinal axis of the cutting member 642, 644). It can be undesirable to have the shear angle change during a cutting procedure, as changes in shear angle can impact the quality or consistency of the cut. In conventional surgical scissors that are not mounted to a robotically-driven surgical instrument (e.g., instrument 630 or any of the other instruments described herein), the shear angle is maintained by nature of the geometry of the blades. In particular, such conventional scissors can have blades that are constrained in translation and axial rotation by relying on blade deformation to press the two blades against each other. The blades can be coupled at a pivot point, and have a proximal segment that is much longer, thereby enabling blade deformation. In robotically-operated surgical scissors, such as the surgical scissors 640 depicted in FIGS. 14A-14C, a proximal length of the cutting members 642, 644 is limited. Therefore, other mechanisms must be used to constrain the axial rotation of the cutting members 642, 644.

FIG. 16 depicts the movements of the cutting members 642, 644 of the surgical scissor 630 in a three-dimensional view. A plurality of axes (x-axis, y-axis, and z-axis) are defined relative to an axis of the pin 641. The x-axis (e.g., a first axis) may extend along a length of the pin 641, such that the x-axis defines the axis of rotation about which the cutting members 642, 644 are configured to rotate (e.g., in a cutting rotation or an opening rotation). Rotation about the x-axis corresponds to the pitch degree of freedom. In embodiments, the cutting members 642, 644 can be configured to pitch between about −110 and about +110 degrees (i.e., pitch downwards from the y-axis to about 110 degrees and pitch upwards from the y-axis to about 110 degrees), inclusive of all values and sub-ranges therebetween, including, for example, between about −90 and about 90 degrees. Each cutting member 642, 644 can be configured to translate along the x-axis. In embodiments, the cutting members 642, 644 can be configured to translate between about 0.1 mm to about 0.5 mm, inclusive of all values and sub-ranges therebetween, including, for example, between about 0.1 mm and 0.2 mm.

The z-axis (e.g., second axis) is perpendicular to the x-axis. In some embodiments, each cutting member 642, 644 can be configured to rotate about the z-axis away from the y-axis as the cutting members 642, 644 move from an open configuration (e.g., as shown in FIG. 15B) to a closed configuration (e.g., as shown in FIG. 15A). In such embodiments, each cutting member 642, 644 can be configured to rotate about the z-axis by an angle of between about 5 and about 15 degrees, inclusive of all values and sub-ranges therebetween, including, for example, between about 7 and about 9 degrees, or about 8.2 degrees. In some embodiments, a single one of the cutting members 642, 644 is configured to rotate about the z-axis as the cutting members 642, 644 move from an open configuration (e.g., as shown in FIG. 15B) to a closed configuration (e.g., as shown in FIG. 15A). For example, a first cutting member 642 can be configured to rotate about the z-axis as the cutting members 642, 644 move from the open configuration to the closed configuration, while the other cutting member 644 does not rotate about the z-axis. In such embodiments, the cutting member 642, 644 that is configured to rotate about the z-axis may be configured to rotate twice as much (e.g., between about 10 and about 30 degrees, inclusive of all values and sub-ranges therebetween), when compared to embodiments where both cutting members 642, 644 can rotate about the z-axis. According to some embodiments, the cutting members 642, 644 may be configured to rotate about the x-axis and/or z-axis and translate along the x-axis simultaneously or as the cutting members 642, 644 move from an open configuration (e.g., as shown in FIG. 15B) to a closed configuration (e.g., as shown in FIG. 15A).

The y-axis (e.g., a third axis) may be perpendicular to the x-axis and z-axis. The y-axis may correspond to a longitudinal length or axis of the cutting members 642, 644. As described above, it can be undesirable to allow for rotation about the y-axis (or axial rotation), as such rotation can impact the shear angle of the cutting members 642, 644. The shear angle, or angle between the inner facing surfaces 642a, 644a of the cutting members 642, 644 at the contact point between the cutting members 642, 644, can affect the quality or consistency of the surgical scissor 630 in performing a cut. Accordingly, maintaining a constant shear angle facilitates consistent cuts, which advantageously results in even cutting performances during use in medical procedures. In embodiments, the shear angle may be between about 160 degrees to about 170 degrees, between about 155 degrees to about 180 degrees, between about 130 and about 180 degrees, inclusive of all sub-ranges and values therebetween.

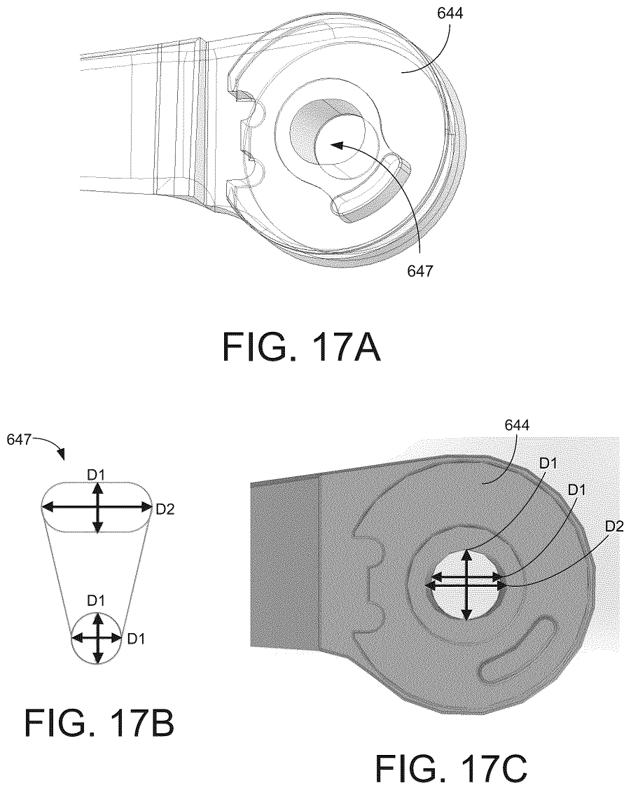

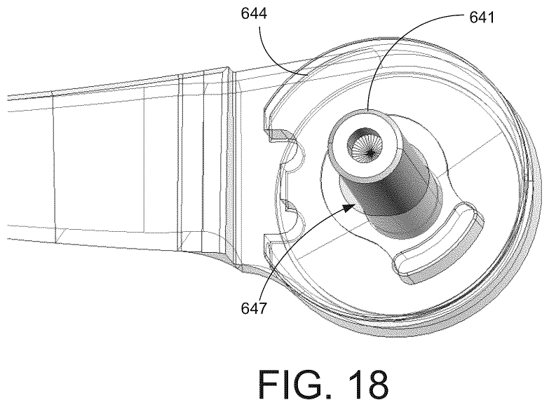

Advantageously, the surgical scissors 630 can be configured to prevent or constrain rotation about the y-axis. In some embodiments, this can be achieved using specifically shaped openings in the mounting bodies of the cutting members 642, 644. For example, each mounting body of the cutting members 642, 644 may comprise an opening configured to allow rotation about one or more of the x- and z-axes, while constraining rotation about the y-axis. Further details of such openings are described in more detail in reference to FIGS. 17A-18.

FIGS. 17A and 17C depict a single cutting member 644 of the surgical scissor 630, showing a hole 647. The hole 647 may define a pivot joint about which the cutting members described herein may pivot. For example, the hole 647 can function as a sliding gimbal (e.g., cardan) configured to allow rotation about one or more axes (e.g., the x- and z-axes) and translation along an axis (e.g., the x-axis), while limiting rotation about another axis (e.g., the y-axis). In some embodiments, the hole 647 can be formed of two openings, e.g., a first opening having a circular shape, and a second opening having an oblong shape. In particular, a first end of the hole 647 may comprise a circular opening and a second end of the hole 647 may comprise an oblong opening, as schematically depicted in FIG. 17B. The circular opening can have a diameter D1 that is substantially the same as or slightly greater than the diameter of the pin. Substantially the same or equal to may refer to dimensions within 10% of each other. In embodiments, being slightly greater than the diameter of the pin is being less than about 10% greater than the diameter of the pin. The oblong opening can have a first lateral dimension that is equal to or substantially equal to the diameter D1 and a second lateral dimension that is greater than the first dimension (i.e., D2, which is greater than D1). In embodiments, a ratio of the first lateral dimension to the second lateral dimension of the first and second oblong openings (D1:D2) is configured to allow a rotation around the z-axis to accommodate the sliding of the contact point between the two first and second cutting members. In some embodiments, the ratio of the first lateral dimension to the second lateral dimension of the first and second oblong openings (D1:D2) is between about 1:1.1 and about 1:1.3, including all values and sub-ranges therebetween.

As shown in FIGS. 17A and 18, the circular and oblong openings may be connected, such that there is a transition from the circular opening to the oblong opening. In some embodiments, the transition may be a gradual transmission. A gradual transmission may be desirable to prevent sharp engagements between the pin surface and the hole 647. While only a single cutting member 644 is depicted in FIGS. 17A and 17C, it can be appreciated that a similar hole can be formed in the mounting body of the other cutting member 642.

When the cutting members 642, 644 are assembled on the pin 641, the oblong openings of the holes 647 of the cutting members 642, 644 may be facing inwards. In other words, the oblong openings of the holes 647 of the cutting members 642, 644 can be positioned to face one another, while the circular openings of the holes 647 of the cutting members 642, 644 can face outwards (e.g., toward the springs 643, 645, respectively). The oblong openings of the holes 647 of the cutting members 642, 644 can be oriented such that the first (or smaller) dimension extends along or is aligned with the z-axis. The second (or larger) dimension can be in the direction of the longitudinal axis of each cutting member 642, 644. When assembled as such, the holes 647 of the cutting members 642, 644 enable rotation about the x- and z-axes and translation about the x-axis, while preventing or constraining or inhibiting rotation about the y-axis. Each cutting member 642, 644 may be constrained in rotation about the y-axis because of the first (or smaller) dimension of the oblong opening being equal to (or substantially equal to) the diameter of the pin 641. Advantageously, the constrained rotation about the y-axis may maintain a constant shear angle, as described previously. The second (larger) dimension of the cutting members 642, 644 can be selected to allow for at least between about 5 and about 15 degrees of rotation around the z-axis, which may accommodate sliding (e.g., translation) of the contact point between the cutting members without creating significant stresses and/or causing failure.

FIG. 18 depicts the pin 641 depicts in the hole 647 of the cutting member 644. The pin 641 may be similarly disposed within a hole of the cutting member 642. As described above, the outer diameter of the pin 641 may be substantially equal to the first lateral dimension of the oblong opening or a diameter of the circular opening. Substantially equal may refer to dimensions within 10% of each other. For example, the outer diameter of the pin 641 may be slightly less than (e.g., within about 10% of) the first lateral dimension of the oblong and/or the diameter of the circular opening, such that the cutting members 642, 644 can rotate about the pin without significant friction and/or griding between components. The relative dimensions of the pin 641 and opening 647 may correspond to a tolerance of a manufacturing process used to manufacture the end effector described herein. In embodiments, the diameter of the pin 641 may be between about 1 mm to about 3 mm, including all values and sub-ranges, or about 1.5 mm.

In some embodiments, surgical scissors as described herein may also include a curvature along a cutting edge of the cutting members to enable an opening angle of the cutting members to remain constant or substantially constant during a cutting rotation. In conventional robotically-operated surgical scissors, the opening angle of the scissor may decrease as the cutting members are closed. For example, FIG. 19A shows an end effector implemented as a surgical scissor 740 in a first configuration where the cutting members 742, 744 of the surgical scissor 740 are near the start of a cutting rotation, and FIG. 19B shows the end effector 740 in a second configuration where the cutting members of the end effector 740 are near the end of the cutting rotation. Each cutting member 742, 744 includes a cutting edge 742a, 744a, respectively. As described before with reference to FIG. 14B, an opening angle may be defined between the cutting edges 742a, 744a at the point of contact therebetween.

As shown in FIGS. 19A and 19B, when the cutting members 742, 744 are further apart (FIG. 19A), the opening angle α1 is greater than the opening angle α2 when the cutting members 742, 744 are closer together (FIG. 19B). The change in opening angles from α1 to α2 may correspond to a change in a slice-push ratio of the end effector 740. The slice-push ratio can refer to the magnitude of cutting a material between the scissor blades to the magnitude of pushing the material between the scissor blades. Generally, a greater opening angle corresponds to a smaller or more optimal slice-push ratio, which may provide more even and/or effective cuts. Therefore, a increasing slice-push ratio, such as the increasing slice-push ratio corresponding to the decrease in opening angles from α1 to α2, may result in less even and/or effective cuts. A varying slice-push ratio, such as a increasing slice-push ratio, may further result in unpredictable cuts. In some cases, the material being cut may become pushed and subsequently compressed (e.g., bunched together) by the cutting edges 742a, 744a such that the resulting cut may be jagged.

In contrast, a surgical scissor as disclosed herein can be configured to maintain larger opening angles during a cutting operation. For example, FIGS. 20A and 20B show an end effector implemented as a surgical scissor 840 in a first and second configurations, respectively, that may both correspond to having the same or substantially the same opening angle α1. In embodiments, the opening angle α1 may be between about 30 degrees to about 40 degrees, about 20 degrees to about 40 degrees, or about 10 degrees to about 50 degrees, inclusive of all values and sub-ranges therebetween. The surgical scissor 840 can be structurally and/or functionally similar to other end effectors and surgical scissors described herein, including, for example, end effector 1040 and/or surgical scissor 640. For example, the surgical scissor 840 can include first and second cutting members 842, 844. In some embodiments, the same opening angle α1 may be achieved by having each cutting member 842, 844 of the surgical scissors 840 have regions with different curvatures. For example, the first cutting member 842 can include a proximal portion 842a and a distal portion 842b, and the second cutting member can include a proximal portion 844a and a distal portion 844b. The proximal portions 842a, 844a of each of the cutting members 842, 844 may comprise a curvature having a first radius of curvature. The distal portions 842b, 844b of each of the cutting members 842, 844 may comprise a curvature having a second radius of curvature, where the second radius of curvature may be smaller than the first radius of curvature.

The distal portion 842b, 844b of each respective cutting member 842, 844 may comprise between about 24% to about 40%, about 20% to about 45%, or about 10% and about 50% of a total length of the respective cutting member 842, 844, including all values and sub-ranges therebetween. Given the change in curvature from the proximal to the distal portions of the first and second cutting members 842, 844, the opening angle may remain constant or substantially constant as the free distal ends of the respective cutting members 842, 844 are rotated toward one another. The movement of the free distal ends towards one another during rotation of the cutting members 842, 844 may form an incision in a cutting plane. As shown in FIGS. 20A and 20B, the curvatures of each of the proximal and distal portions of each respective cutting member 842, 844 curve in a direction away from the cutting plane. In some embodiments, the cutting members 842, 844 may also include one or more additional curvatures in other directions, e.g., in a direction along the cutting plane.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto; inventive embodiments may be practiced otherwise than as specifically described and claimed. Inventive embodiments of the present disclosure are directed to each individual feature, system, article, material, and/or method described herein. In addition, any combination of two or more such features, systems, articles, materials, and/or methods, if such features, systems, articles, materials, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

As used herein, the terms “about,” “approximately,” and/or “substantially” when used in connection with stated value(s), geometric structure(s), relationship(s), or other characteristic(s) is intended to convey that the value or characteristic so defined is nominally the value stated or characteristic described. In some instances, the terms “about,” “approximately,” and/or “substantially” can generally mean and/or can generally contemplate a value or characteristic stated within a desirable tolerance, e.g., plus or minus 10% of the value or characteristic stated. For example, a value of about 0.01 can include 0.009 and 0.011, a value of about 0.5 can include 0.45 and 0.55, a value of about 10 can include 9 to 11, and a value of about 1000 can include 900 to 1100. Similarly, a value or characteristic may be described as being substantially constant when the value or characteristic does not change by more than about 10%. While a value, structure, and/or relationship stated may be desirable, it should be understood that some variance may occur as a result of, for example, manufacturing tolerances or other practical considerations (such as, for example, the pressure or force applied through a portion of a device). Accordingly, the terms “about,” “approximately,” and/or “substantially” can be used herein to account for such tolerances and/or considerations.

The indefinite articles “a” and “an,” as used herein in the specification and in the claims, unless clearly indicated to the contrary, should be understood to mean “at least one.”

1. An apparatus, comprising:
a first cutting member and a second cutting member, each of the first and second cutting members comprising a cutting edge and a mounting end defining an oblong opening; and
a pin extending through the oblong openings of each of the mounting ends of the first and second cutting members,
wherein each of the first and second cutting members is configured to independently rotate about a first axis defined by the pin and a second axis perpendicular to the first axis while being constrained in rotation about a third axis perpendicular to the first and second axes.

2. The apparatus of claim 1, wherein each of the first and second cutting members have a curved section.

3. The apparatus of claim 1, wherein the cutting edge of the first cutting member contacts the cutting edge of the second cutting member at a contact point that translates along the respective cutting edges when one or more of the first and second cutting members rotate about the first axis.

4. The apparatus of claim 1, wherein the shear angle is between about 160 degrees to about 180 degrees.

5. The apparatus of claim 1, wherein each oblong opening has a first lateral dimension that is smaller than a second lateral dimension, and the second axis is parallel to the first lateral dimension.

6. The apparatus of claim 5, wherein the first lateral dimension is substantially equal to an outer diameter of the pin.

7. The apparatus of claim 1, further comprising a first spring coupled to the first cutting member and a second spring coupled to the second cutting member, the first and second springs configured to press the first and second cutting members against one another.

8. The apparatus of claim 7, wherein each of the first and second springs comprises a Belleville spring.

9. The apparatus of claim 7, wherein each of the first and second springs is configured to allow the respective first and second cutting members to translate along the first axis.

10. The apparatus of claim 1, wherein the first and second cutting members are configured to translate along the first axis.

11. The apparatus of claim 1, wherein each of the mounting ends of the first and second cutting members further defines a circular opening that is connected to the oblong opening,
the pin configured to extend through the circular opening and the oblong opening of each of the mounting ends of the first and second cutting members.

12. The apparatus of claim 11, wherein each oblong opening has a first lateral dimension that is smaller than a second lateral dimension,
the first lateral dimension of the oblong opening being substantially equal to a diameter of the circular opening.

13. The apparatus of claim 12, wherein the first lateral dimension of the oblong opening and the diameter of the circular opening are substantially equal to an outer diameter of the pin.

14. The apparatus of claim 11, wherein the oblong openings are disposed on inner sides of the first and second cutting members that face one another, and the circular openings are disposed on outer sides of the first and second cutting members.

15. An apparatus, comprising:
a first cutting member comprising a first cutting edge and a first mounting end defining a first oblong opening;
a second cutting member comprising a second cutting edge and a second mounting end defining a second oblong opening aligned with the first oblong opening; and
a cylindrical pin defining an x-axis, the cylindrical pin extending through the first and second oblong openings,
wherein each of the first and second oblong openings includes a first lateral dimension substantially equal to a diameter of the cylindrical pin and a second lateral dimension greater than the diameter of the cylindrical pin,
wherein each of the first and second cutting members is configured to (1) independently rotate about the x-axis and about a z-axis that is parallel to the first lateral dimension of the first and second oblong openings and (2) independently translate along the x-axis, while being constrained in rotation about a y-axis that is perpendicular to the x-axis and the z-axis.

16. The apparatus of claim 15, wherein each of the first and second cutting members have a curved section.

17. The apparatus of claim 15, wherein a ratio of the first lateral dimension to the second lateral dimension of the first and second oblong openings is configured to allow a rotation around the z-axis to accommodate a sliding of the contact point between the first and second cutting members.

18. The apparatus of claim 15, wherein a ratio of the first lateral dimension to the second lateral dimension of the first and second oblong openings is between about 1:1.1 and about 1:1.3.

19. The apparatus of claim 15, wherein each of the first and second cutting members is configured to rotate about one or more of the x-axis and the z-axis and translate along the x-axis simultaneously.

20. The apparatus of claim 15, further comprising a first spring coupled to the first cutting member and a second spring coupled to the second cutting member, the first and second springs configured to press the first and second cutting members against one another.

21. The apparatus of claim 20, wherein each of the first and second springs comprises a Belleville spring.

22. The apparatus of claim 15, wherein the first cutting edge contacts the second cutting edge at a contact point that translates along the first and second cutting edges when one or more of the first and second cutting members rotate about the x-axis.

23.-62. (canceled)
